SEQUENCE LISTING (13 sequences)

SEQ ID NO: 1 - protein, 538 aa, Murine leukemia virus
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLQYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDRDQCAYCKEKGHWAKDCPKKPRGPRGPRPQTSLLTLDD

SEQ ID NO: 2 - DNA, 1614 bp, Murine leukemia virus
  source 1..1614: mol_type = genomic DNA
atgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgac

SEQ ID NO: 3 - DNA, 1614 bp, synthetic construct
  misc_feature 1..1614: note = Codon optimised
  source 1..1614: mol_type = other DNA
atgggacagaccgtcacaacacccctgagcctgaccctgggacattggaaagacgtggagaggatcgcacataaccagagcgtggacgtgaagaaacggagatgggtcacattctgcagtgctgagtggccaacttttaatgtgggatggccccgagacggcactttcaacagggatctgatcacccaggtgaagatcaaggtctttagcccaggacctcacggacatccagaccaggtgccttatatcgtcacctgggaggcactggccttcgatccccctccatgggtgaagccatttgtccacccaaaaccacctccaccactgcctccaagtgccccttcactgccactggaaccaccccggagcacaccaccccgcagctccctgtatcctgctctgactccatctctgggcgcaaagccaaaaccacaggtgctgagcgactccggaggaccactgattgacctgctgacagaggaccccccaccataccgagatcctcggcctccaccaagcgaccgcgatggaaatggaggagaggctactcctgccggcgaagcccctgacccatctccaatggctagtaggctgcgcggcaggcgcgagcctccagtggcagatagcaccacatcccaggccttccctctgagggctgggggaaatgggcagctccagtattggccattttctagttcagacctgtacaactggaagaacaataacccctctttcagtgaggaccccggcaaactgaccgccctgatcgaatccgtgctgattacccatcagcccacatgggacgattgtcagcagctcctgggcaccctgctgaccggagaggaaaagcagcgcgtgctgctggaggctcgcaaagcagtccgaggggacgatggacggcccacacagctccctaatgaggtggacgccgcttttccactggaaagacccgactgggattatactacccaggcagggagaaaccacctggtccattacaggcagctcctgctggcaggcctgcagaatgccgggagatcccccaccaacctggccaaggtgaaaggcatcacacaggggcctaatgagtcaccaagcgcctttctggagaggctgaaggaagcttaccgacggtataccccatacgaccctgaggaccccggacaggaaacaaacgtctccatgtctttcatctggcagtctgccccagacattgggcggaagctggagagactggaagacctgaagaacaagaccctgggcgacctggtgcgggaggctgaaaagatcttcaacaaacgggagacccccgaggaaagagaggaaaggattagaagggaaactgaggaaaaggaggaacgccgacggaccgaggacgaacagaaggagaaagaacgagatcggcggcggcaccgggagatgtcaaagctgctggccaccgtggtcagcggacagaaacaggacagacagggaggagagcgacggagaagccagctcgacagggatcagtgcgcatactgtaaggaaaaaggccattgggccaaggattgccccaaaaagccaagaggaccaagaggaccaagaccacagacatcactgctgaccctggacgac

SEQ ID NO: 4 - protein, 1099 aa, synthetic construct
  REGION 1..1099: note = MMLV Gag - CMV pp65 Amino Acid Sequence
  REGION 1..538: note = MMLV Gag amino acid sequence
  REGION 539..1099: note = CMV pp65 Amino Acid Sequence
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLQYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDRDQCAYCKEKGHWAKDCPKKPRGPRGPRPQTSLLTLDDCESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRKRKSASSATACTAGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 5 - DNA, 3300 bp, synthetic construct
  misc_feature 1..3300: note = MMLV Gag - CMV pp65 Nucleotide Sequence
  misc_feature 1..1614: note = MMLV Gag nucleotide sequence
  misc_feature 1615..3297: note = CMV pp65 nucleotide sequence
  misc_feature 3298..3300: note = stop
  source 1..3300: mol_type = other DNA
atgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactgtgagtcgcgcggtcgccgttgtcccgaaatgatatccgtactgggtcccatttcggggcacgtgctgaaagccgtgtttagtcgcggcgacacgccggtgctgccgcacgagacgcgactcctgcagacgggtatccacgtgcgcgtgagccagccctcgctgatcctggtgtcgcagtacacgcccgactcgacgccatgccaccgcggcgacaatcagctgcaggtgcagcacacgtactttacgggcagcgaggtggagaacgtgtcggtcaacgtgcacaaccccacgggccggagcatctgccccagccaagagcccatgtcgatctatgtgtacgcgctgccgctcaagatgctgaacatccccagcatcaacgtgcaccactacccgtcggcggccgagcgcaaacaccgacacctgcccgtagctgacgctgtgattcacgcgtcgggcaagcagatgtggcaggcgcgtctcacggtctcgggactggcctggacgcgtcagcagaaccagtggaaagagcccgacgtctactacacgtcagcgttcgtgtttcccaccaaggacgtggcactgcggcacgtggtgtgcgcgcacgagctggtttgctccatggagaacacgcgcgcaaccaagatgcaggtgataggtgaccagtacgtcaaggtgtacctggagtccttctgcgaggacgtgccctccggcaagctctttatgcacgtcacgctgggctctgacgtggaagaggacctgacgatgacccgcaacccgcaacccttcatgcgcccccacgagcgcaacggctttacggtgttgtgtcccaaaaatatgataatcaaaccgggcaagatctcgcacatcatgctggatgtggcttttacctcacacgagcattttgggctgctgtgtcccaagagcatcccgggcctgagcatctcaggtaacctattgatgaacgggcagcagatcttcctggaggtgcaagcgatacgcgagaccgtggaactgcgtcagtacgatcccgtggctgcgctcttctttttcgatatcgacttgctgctgcagcgcgggcctcagtacagcgaacaccccaccttcaccagccagtatcgcatccagggcaagcttgagtaccgacacacctgggaccggcacgacgagggtgccgcccagggcgacgacgacgtctggaccagcggatcggactccgacgaggaactcgtaaccaccgagcgcaagacgccccgcgttaccggcggcggcgccatggcgggcgcctccacttccgcgggccgcaaacgcaaatcagcatcctcggcgacggcgtgcacggcgggcgttatgacacgcggccgccttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggagttcttctgggacgccaacgacatctaccgcatcttcgccgaattggaaggcgtatggcagcccgctgcgcaacccaaacgtcgccgccaccggcaagacgccttgcccgggccatgcatcgcctcgacgcccaaaaagcaccgaggttag

SEQ ID NO: 6 - DNA, 3300 bp, synthetic construct
  misc_feature 1..3300: note = Codon Optimized MMLV Gag - CMV pp65 Nucleotide Sequence
  misc_feature 1..1614: note = Codon Optimized MMLV Gag
  misc_feature 1615..3297: note = Codon Optimized CMV pp65
  misc_feature 3298..3300: note = stop
  source 1..3300: mol_type = other DNA
atgggacagacagtcactacacccctgagcctgacactgggacattggaaagacgtggagaggattgcacataaccagagcgtggacgtgaagaaacggagatgggtcaccttttgctccgccgagtggccaacattcaatgtgggatggccccgagatggcaccttcaaccgggacctgatcactcaggtgaagatcaaggtcttctctccaggaccccacggccatccagatcaggtgccctacatcgtcacctgggaggctctggcatttgacccccctccatgggtgaagcctttcgtccacccaaaaccacctccaccactgcctccatctgcccctagtctgccactggaaccccctcggtcaaccccacccagaagctccctgtatcccgcactgacacctagcctgggggccaagcctaaaccacaggtgctgtctgatagtggcgggcctctgatcgatctgctgaccgaggaccctccaccataccgcgacccacgacctccaccaagcgaccgggacggaaacggaggagaggctacacccgcaggcgaagcccccgatcctagtccaatggcatcaaggctgcgcgggaggcgcgaacctccagtggccgactcaaccacaagccaggcatttccactgagggccgggggaaatggacagctccagtattggcccttctctagttcagatctgtacaactggaagaacaataaccctagcttcagcgaggacccaggcaaactgaccgccctgatcgaatccgtgctgattacccaccagcccacatgggacgattgtcagcagctcctgggcaccctgctgaccggagaggaaaagcagagagtgctgctggaggctaggaaagcagtccgcggggacgatggaaggccaacacagctccccaatgaggtggatgccgctttccctctggaacggccagattgggactatactacccaggctggacgcaaccacctggtgcattaccggcagctcctgctggctggactgcagaatgcagggcgcagccccactaacctggccaaggtgaaaggaatcacccagggccccaatgagtccccttctgcattcctggagcggctgaaggaagcctaccgacggtatactccctacgatcctgaggacccaggccaggaaaccaacgtgagtatgagcttcatctggcagtccgctcctgacattggccgaaaactggagcggctggaagatctgaagaacaagaccctgggcgacctggtgcgggaggcagaaaagatcttcaacaaaagggagactccagaggaacgggaggaaagaattagaagggaaacagaggaaaaggaggaacgccgacggactgaggatgaacagaaggagaaagaaagagaccggcggcggcaccgggagatgtctaagctgctggccaccgtggtcagtggccagaaacaggatcgacagggaggagagcgacggagaagccagctcgatcgggaccagtgcgcctattgtaaggaaaaagggcattgggctaaggactgccccaagaaacccagaggcccacgcgggccccgacctcagacttccctgctgaccctggacgattgcgagagccggggccggcggtgcccagaaatgatctctgtgctggggcccattagtggacatgtgctgaaggccgtcttctccaggggagacacccccgtgctgcctcacgagactcgactgctgcagaccggcatccatgtgcgggtctcccagccctctctgattctggtgtcacagtatacaccagatagcactccctgccacagaggagacaatcagctccaggtgcagcatacctactttacaggctccgaggtcgaaaacgtgtctgtcaatgtgcacaaccctaccggcaggagcatctgtcctagccaggagccaatgagcatctacgtgtacgccctgcctctgaagatgctgaatatcccatcaattaacgtccaccattaccctagcgcagccgaacggaagcacagacatctgccagtggccgacgctgtcatccatgccagcggcaaacagatgtggcaggcaagactgaccgtgtccgggctggcctggacaaggcagcagaatcagtggaaggagcccgacgtgtactataccagcgccttcgtgttccctaccaaagacgtggccctgagacatgtggtgtgcgcacatgagctggtgtgcagcatggaaaacactagggccaccaagatgcaggtcatcggcgatcagtatgtcaaagtgtacctggagagtttttgcgaagacgtgccatcagggaagctgttcatgcatgtgaccctgggcagcgatgtcgaggaagacctgaccatgacaagaaatccacagccctttatgagaccccacgagaggaatgggttcactgtgctgtgccccaagaacatgatcattaagcctggaaaaatcagtcatattatgctggatgtggcctttacatcacacgagcatttcggactgctgtgccccaaatccatccctggactgagcatttccggcaatctgctgatgaacggccagcagatcttcctggaagtgcaggccatccgggagaccgtcgaactgcgacagtatgacccagtggctgcactgttctttttcgacatcgacctgctgctgcagcgaggaccacagtacagcgagcaccctacttttacctcccagtatcggattcaggggaagctggagtacaggcacacctgggatcgccatgacgaaggagccgctcagggggacgatgacgtgtggacatctggcagtgattcagacgaggaactggtgacaactgagcgaaaaaccccccgggtgacaggaggaggggcaatggcaggggccagcaccagcgcagggcggaagcgaaaaagcgccagcagcgccacagcatgtaccgccggcgtgatgactagaggaaggctgaaggccgagtctacagtcgctcccgaggaagatactgacgaggatagtgacaatgaaatccacaaccccgccgtgttcacctggccaccttggcaggcagggattctggctcgcaacctggtccccatggtggcaaccgtccagggacagaatctgaagtatcaggagtttttctgggatgctaacgacatctaccggatttttgcagagctggaaggcgtgtggcagccagcagcccagcccaaacgacggagacatcgacaggacgctctgccaggaccttgtatcgccagcacaccaaagaagcacaggggctaa

SEQ ID NO: 7 - DNA, 3300 bp, synthetic construct
  misc_feature 1..3300: note = Codon Optimized MMLV Gag - CMV pp65 Nucleotide Sequence
  misc_feature 1..1614: note = Codon Optimized MMLV Gag
  misc_feature 1615..3297: note = Codon Optimized CMV pp65
  misc_feature 3298..3300: note = stop
  source 1..3300: mol_type = other DNA
atgggacagaccgtcacaacacccctgagcctgaccctgggacattggaaagacgtggagaggatcgcacataaccagagcgtggacgtgaagaaacggagatgggtcacattctgcagtgctgagtggccaacttttaatgtgggatggccccgagacggcactttcaacagggatctgatcacccaggtgaagatcaaggtctttagcccaggacctcacggacatccagaccaggtgccttatatcgtcacctgggaggcactggccttcgatccccctccatgggtgaagccatttgtccacccaaaaccacctccaccactgcctccaagtgccccttcactgccactggaaccaccccggagcacaccaccccgcagctccctgtatcctgctctgactccatctctgggcgcaaagccaaaaccacaggtgctgagcgactccggaggaccactgattgacctgctgacagaggaccccccaccataccgagatcctcggcctccaccaagcgaccgcgatggaaatggaggagaggctactcctgccggcgaagcccctgacccatctccaatggctagtaggctgcgcggcaggcgcgagcctccagtggcagatagcaccacatcccaggccttccctctgagggctgggggaaatgggcagctccagtattggccattttctagttcagacctgtacaactggaagaacaataacccctctttcagtgaggaccccggcaaactgaccgccctgatcgaatccgtgctgattacccatcagcccacatgggacgattgtcagcagctcctgggcaccctgctgaccggagaggaaaagcagcgcgtgctgctggaggctcgcaaagcagtccgaggggacgatggacggcccacacagctccctaatgaggtggacgccgcttttccactggaaagacccgactgggattatactacccaggcagggagaaaccacctggtccattacaggcagctcctgctggcaggcctgcagaatgccgggagatcccccaccaacctggccaaggtgaaaggcatcacacaggggcctaatgagtcaccaagcgcctttctggagaggctgaaggaagcttaccgacggtataccccatacgaccctgaggaccccggacaggaaacaaacgtctccatgtctttcatctggcagtctgccccagacattgggcggaagctggagagactggaagacctgaagaacaagaccctgggcgacctggtgcgggaggctgaaaagatcttcaacaaacgggagacccccgaggaaagagaggaaaggattagaagggaaactgaggaaaaggaggaacgccgacggaccgaggacgaacagaaggagaaagaacgagatcggcggcggcaccgggagatgtcaaagctgctggccaccgtggtcagcggacagaaacaggacagacagggaggagagcgacggagaagccagctcgacagggatcagtgcgcatactgtaaggaaaaaggccattgggccaaggattgccccaaaaagccaagaggaccaagaggaccaagaccacagacatcactgctgaccctggacgactgcgagagccggggccggcggtgcccagaaatgatctctgtgctggggcccattagtggacatgtgctgaaggccgtcttctccaggggagacacccccgtgctgcctcacgagactcgactgctgcagaccggcatccatgtgcgggtctcccagccctctctgattctggtgtcacagtatacaccagatagcactccctgccacagaggagacaatcagctccaggtgcagcatacctactttacaggctccgaggtcgaaaacgtgtctgtcaatgtgcacaaccctaccggcaggagcatctgtcctagccaggagccaatgagcatctacgtgtacgccctgcctctgaagatgctgaatatcccatcaattaacgtccaccattaccctagcgcagccgaacggaagcacagacatctgccagtggccgacgctgtcatccatgccagcggcaaacagatgtggcaggcaagactgaccgtgtccgggctggcctggacaaggcagcagaatcagtggaaggagcccgacgtgtactataccagcgccttcgtgttccctaccaaagacgtggccctgagacatgtggtgtgcgcacatgagctggtgtgcagcatggaaaacactagggccaccaagatgcaggtcatcggcgatcagtatgtcaaagtgtacctggagagtttttgcgaagacgtgccatcagggaagctgttcatgcatgtgaccctgggcagcgatgtcgaggaagacctgaccatgacaagaaatccacagccctttatgagaccccacgagaggaatgggttcactgtgctgtgccccaagaacatgatcattaagcctggaaaaatcagtcatattatgctggatgtggcctttacatcacacgagcatttcggactgctgtgccccaaatccatccctggactgagcatttccggcaatctgctgatgaacggccagcagatcttcctggaagtgcaggccatccgggagaccgtcgaactgcgacagtatgacccagtggctgcactgttctttttcgacatcgacctgctgctgcagcgaggaccacagtacagcgagcaccctacttttacctcccagtatcggattcaggggaagctggagtacaggcacacctgggatcgccatgacgaaggagccgctcagggggacgatgacgtgtggacatctggcagtgattcagacgaggaactggtgacaactgagcgaaaaaccccccgggtgacaggaggaggggcaatggcaggggccagcaccagcgcagggcggaagcgaaaaagcgccagcagcgccacagcatgtaccgccggcgtgatgactagaggaaggctgaaggccgagtctacagtcgctcccgaggaagatactgacgaggatagtgacaatgaaatccacaaccccgccgtgttcacctggccaccttggcaggcagggattctggctcgcaacctggtccccatggtggcaaccgtccagggacagaatctgaagtatcaggagtttttctgggatgctaacgacatctaccggatttttgcagagctggaaggcgtgtggcagccagcagcccagcccaaacgacggagacatcgacaggacgctctgccaggaccttgtatcgccagcacaccaaagaagcacaggggctaa

SEQ ID NO: 8 - protein, 907 aa, Human cytomegalovirus
  REGION 752..907: note = TM and CD
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSINVFDLEEIMREFNSYKQRVKYVEDKVVDPLPPYLKGLDDLMSGLGAAGKAVGVAIGAVGGAVASVVEGVATFLKNPFGAFTIILVAIAVVIIIYLIYTRQRRLCMQPLQNLFPYLVSADGTTVTSGNTKDTSLQAPPSYEESVYNSGRKGPGPPSSDASTAAPPYTNEQAYQMLLALVRLDAEQRAQQNGTDSLDGQTGTQDKGQKPNLLDRLRHRKNGYRHLKDSDEEENV

SEQ ID NO: 9 - DNA, 2724 bp, Human cytomegalovirus
  misc_feature 2254..2721: note = TM and CD
  misc_feature 2722..2724: note = stop
  source 1..2724: mol_type = genomic DNA
atggagtcaaggatttggtgcctggtcgtgtgcgtcaatctgtgcatcgtctgtctgggggctgccgtgtcatcaagttctacaagaggcaccagcgccacccactcacaccatagctcccataccacatccgccgctcactcccggtctggcagcgtgagccagagagtcacatctagtcagaccgtgagccacggggtcaacgagaccatctacaatactaccctgaagtatggcgacgtggtcggggtgaacacaactaaatacccatatagggtctgcagtatggcccagggcactgatctgattagattcgaaaggaacatcgtgtgcaccagcatgaagcccattaatgaggacctggatgaagggatcatggtggtctacaaacgcaatattgtggcccataccttcaaggtgcgagtctatcagaaagtgctgacatttcggagatcttacgcatatatccacaccacatacctgctggggagtaacaccgagtatgtggctccccctatgtgggaaattcaccatatcaatagccattcccagtgctactcaagctacagcagagtgatcgctggaacagtgttcgtcgcataccacagagactcttatgagaacaagactatgcagctcatgcccgacgattacagcaatacacattccactagatatgtgacagtcaaagatcagtggcactcaaggggcagcacctggctgtaccgcgagacatgcaacctgaattgtatggtgactatcactaccgctagatccaagtacccctatcacttctttgcaacttccaccggggacgtggtcgatatttctcctttctacaacggcacaaaccggaatgcatcttattttggggagaacgccgacaagttctttattttcccaaattacaccatcgtgtctgattttggcagacccaacagtgccctggagacacatcgactggtggcattcctggaacgggccgactccgtcatttcttgggacatccaggatgagaagaatgtgacctgccagctcaccttctgggaggccagcgaacgcaccatccgatccgaggctgaagattcttaccacttctcctctgccaaaatgacagctacttttctgagcaagaaacaggaggtgaacatgtctgacagtgctctggattgcgtgcgggacgaagcaattaataagctgcagcagatcttcaacacatcatacaaccagacttacgagaagtacggaaacgtgagcgtcttcgaaacaactggcgggctggtggtcttttggcagggcatcaagcagaaatccctggtggagctggaaaggctggccaatcgcagttcactgaacctgactcataatcggaccaagagatctacagacggaaacaatgccacacatctgtctaacatggagagtgtgcacaatctggtctacgctcagctccagtttacctacgacacactgagaggctatattaacagggcactggcccagatcgctgaagcatggtgcgtggatcagaggcgcaccctggaggtcttcaaggaactgtccaaaatcaacccttcagcaattctgagcgccatctacaataagccaattgcagccaggtttatgggagacgtgctgggcctggccagttgcgtcactatcaaccagacctcagtgaaggtcctgcgcgatatgaatgtgaaagagagtcccggcagatgctattcacggcctgtggtcatcttcaactttgctaatagctcctacgtgcagtatggacagctcggcgaggacaacgaaattctgctggggaatcacaggaccgaggaatgtcagctccctagcctgaagattttcatcgctggaaactccgcatacgagtatgtggattacctgttcaagcggatgattgacctgtctagtatctccactgtggattctatgattgccctggacatcgatccactggaaaataccgacttcagggtgctggagctgtatagccagaaggaactgcgctccatcaacgtgttcgatctggaggaaattatgagagagtttaatagctacaagcagagggtgaaatatgtcgaagataaggtggtcgaccccctgccaccctacctgaaaggcctggacgatctgatgagcgggctgggagctgcagggaaggcagtgggagtcgctatcggcgcagtgggaggagccgtggccagcgtggtcgagggagtggcaacattcctgaaaaaccccttcggggccttcaccatcattctggtggcaatcgccgtggtcatcattatctacctgatctacacaaggcagcggcggctgtgcatgcagcctctgcagaacctgtttccatacctggtgagcgccgacgggaccacagtcacctcaggaaatactaaggatacctctctgcaggcccccccaagttacgaggaatcagtgtataacagcggcagaaaaggaccaggaccaccttcaagcgacgccagcactgccgctccaccctacaccaatgagcaggcctatcagatgctgctggctctggtgcgcctggatgccgaacagcgagctcagcagaacgggaccgactccctggatggacagaccggaacacaggacaagggacagaaacctaatctgctggatcggctgcggcacagaaaaaacgggtataggcacctgaaggactccgacgaagaagaaaatgtctaa

SEQ ID NO: 10 - DNA, 2724 bp, synthetic construct
  misc_feature 1..2724: note = Codon Optimized HCMV gB Nucleotide Sequence
  misc_feature 2254..2721: note = TM and CD
  misc_feature 2722..2724: note = stop
  source 1..2724: mol_type = other DNA
atggaatccaggatctggtgcctggtagtctgcgttaacttgtgtatcgtctgtctgggtgctgcggtttcctcatcttctactcgtggaacttctgctactcacagtcaccattcctctcatacgacgtctgctgctcattctcgatccggttcagtctctcaacgcgtaacttcttcccaaacggtcagccatggtgttaacgagaccatctacaacactaccctcaagtacggagatgtggtgggggtcaacaccaccaagtacccctatcgcgtgtgttctatggcacagggtacggatcttattcgctttgaacgtaatatcgtctgcacctcgatgaagcccatcaatgaagacctggacgagggcatcatggtggtctacaaacgcaacatcgtcgcgcacacctttaaggtacgagtctaccagaaggttttgacgtttcgtcgtagctacgcttacatccacaccacttatctgctgggcagcaacacggaatacgtggcgcctcctatgtgggagattcatcatatcaacagtcacagtcagtgctacagttcctacagccgcgttatagcaggcacggttttcgtggcttatcatagggacagctatgaaaacaaaaccatgcaattaatgcccgacgattattccaacacccacagtacccgttacgtgacggtcaaggatcaatggcacagccgcggcagcacctggctctatcgtgagacctgtaatctgaattgtatggtgaccatcactactgcgcgctccaagtatccctatcattttttcgcaacttccacgggtgatgtggttgacatttctcctttctacaacggaactaatcgcaatgccagctattttggagaaaacgccgacaagtttttcatttttccgaactacactatcgtctccgactttggaagaccgaattctgcgttagagacccacaggttggtggcttttcttgaacgtgcggactcagtgatctcctgggatatacaggacgagaagaatgttacttgtcaactcactttctgggaagcctcggaacgcaccattcgttccgaagccgaggactcgtatcacttttcttctgccaaaatgaccgccactttcttatctaagaagcaagaggtgaacatgtccgactctgcgctggactgtgtacgtgatgaggccataaataagttacagcagattttcaatacttcatacaatcaaacatatgaaaaatatggaaacgtgtccgtctttgaaaccactggtggtttggtggtgttctggcaaggtatcaagcaaaaatctctggtggaactcgaacgtttggccaaccgctccagtctgaatcttactcataatagaaccaaaagaagtacagatggcaacaatgcaactcatttatccaacatggagtcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaacttagcaagatcaacccgtcagctattctctcggccatctacaacaaaccgattgccgcgcgtttcatgggtgatgtcctgggtctggccagctgcgtgaccattaaccaaaccagcgtcaaggtgctgcgtgatatgaatgtgaaggaatcgccaggacgctgctactcacgaccagtggtcatctttaatttcgccaacagctcgtacgtgcagtacggtcaactgggcgaggataacgaaatcctgttgggcaaccaccgcactgaggaatgtcagcttcccagcctcaagatcttcatcgccggcaactcggcctacgagtacgtggactacctcttcaaacgcatgattgacctcagcagcatctccaccgtcgacagcatgatcgccctagacatcgacccgctggaaaacaccgacttcagggtactggaactttactcgcagaaagaattgcgttccatcaacgtttttgatctcgaggagatcatgcgcgagttcaattcgtataagcagcgggtaaagtacgtggaggacaaggtagtcgacccgctgccgccctacctcaagggtctggacgacctcatgagcggcctgggcgccgcgggaaaggccgttggcgtagccattggggccgtgggtggcgcggtggcctccgtggtcgaaggcgttgccaccttcctcaaaaaccccttcggagccttcaccatcatcctcgtggccatagccgtcgtcattatcatttatttgatctatactcgacagcggcgtctctgcatgcagccgctgcagaacctctttccctatctggtgtccgccgacgggaccaccgtgacgtcgggcaacaccaaagacacgtcgttacaggctccgccttcctacgaggaaagtgtttataattctggtcgcaaaggaccgggaccaccgtcgtctgatgcatccacggcggctccgccttacaccaacgagcaggcttaccagatgcttctggccctggtccgtctggacgcagagcagcgagcgcagcagaacggtacagattctttggacggacagactggcacgcaggacaagggacagaagcccaacctgctagaccgactgcgacaccgcaaaaacggctaccgacacttgaaagactccgacgaagaagagaacgtctga

SEQ ID NO: 11 - protein, 561 aa, Human cytomegalovirus
CESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRKRKSASSATACTAGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 12 - DNA, 1683 bp, Human cytomegalovirus
  source 1..1683: mol_type = genomic DNA
tgtgagtcgcgcggtcgccgttgtcccgaaatgatatccgtactgggtcccatttcggggcacgtgctgaaagccgtgtttagtcgcggcgacacgccggtgctgccgcacgagacgcgactcctgcagacgggtatccacgtgcgcgtgagccagccctcgctgatcctggtgtcgcagtacacgcccgactcgacgccatgccaccgcggcgacaatcagctgcaggtgcagcacacgtactttacgggcagcgaggtggagaacgtgtcggtcaacgtgcacaaccccacgggccggagcatctgccccagccaagagcccatgtcgatctatgtgtacgcgctgccgctcaagatgctgaacatccccagcatcaacgtgcaccactacccgtcggcggccgagcgcaaacaccgacacctgcccgtagctgacgctgtgattcacgcgtcgggcaagcagatgtggcaggcgcgtctcacggtctcgggactggcctggacgcgtcagcagaaccagtggaaagagcccgacgtctactacacgtcagcgttcgtgtttcccaccaaggacgtggcactgcggcacgtggtgtgcgcgcacgagctggtttgctccatggagaacacgcgcgcaaccaagatgcaggtgataggtgaccagtacgtcaaggtgtacctggagtccttctgcgaggacgtgccctccggcaagctctttatgcacgtcacgctgggctctgacgtggaagaggacctgacgatgacccgcaacccgcaacccttcatgcgcccccacgagcgcaacggctttacggtgttgtgtcccaaaaatatgataatcaaaccgggcaagatctcgcacatcatgctggatgtggcttttacctcacacgagcattttgggctgctgtgtcccaagagcatcccgggcctgagcatctcaggtaacctattgatgaacgggcagcagatcttcctggaggtgcaagcgatacgcgagaccgtggaactgcgtcagtacgatcccgtggctgcgctcttctttttcgatatcgacttgctgctgcagcgcgggcctcagtacagcgaacaccccaccttcaccagccagtatcgcatccagggcaagcttgagtaccgacacacctgggaccggcacgacgagggtgccgcccagggcgacgacgacgtctggaccagcggatcggactccgacgaggaactcgtaaccaccgagcgcaagacgccccgcgttaccggcggcggcgccatggcgggcgcctccacttccgcgggccgcaaacgcaaatcagcatcctcggcgacggcgtgcacggcgggcgttatgacacgcggccgccttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggagttcttctgggacgccaacgacatctaccgcatcttcgccgaattggaaggcgtatggcagcccgctgcgcaacccaaacgtcgccgccaccggcaagacgccttgcccgggccatgcatcgcctcgacgcccaaaaagcaccgaggt

SEQ ID NO: 13 - DNA, 1683 bp, synthetic construct
  misc_feature 1..1683: note = Codon Optimized HCMV pp65 Nucleotide Sequence
  source 1..1683: mol_type = other DNA
tgcgagagccggggccggcggtgcccagaaatgatctctgtgctggggcccattagtggacatgtgctgaaggccgtcttctccaggggagacacccccgtgctgcctcacgagactcgactgctgcagaccggcatccatgtgcgggtctcccagccctctctgattctggtgtcacagtatacaccagatagcactccctgccacagaggagacaatcagctccaggtgcagcatacctactttacaggctccgaggtcgaaaacgtgtctgtcaatgtgcacaaccctaccggcaggagcatctgtcctagccaggagccaatgagcatctacgtgtacgccctgcctctgaagatgctgaatatcccatcaattaacgtccaccattaccctagcgcagccgaacggaagcacagacatctgccagtggccgacgctgtcatccatgccagcggcaaacagatgtggcaggcaagactgaccgtgtccgggctggcctggacaaggcagcagaatcagtggaaggagcccgacgtgtactataccagcgccttcgtgttccctaccaaagacgtggccctgagacatgtggtgtgcgcacatgagctggtgtgcagcatggaaaacactagggccaccaagatgcaggtcatcggcgatcagtatgtcaaagtgtacctggagagtttttgcgaagacgtgccatcagggaagctgttcatgcatgtgaccctgggcagcgatgtcgaggaagacctgaccatgacaagaaatccacagccctttatgagaccccacgagaggaatgggttcactgtgctgtgccccaagaacatgatcattaagcctggaaaaatcagtcatattatgctggatgtggcctttacatcacacgagcatttcggactgctgtgccccaaatccatccctggactgagcatttccggcaatctgctgatgaacggccagcagatcttcctggaagtgcaggccatccgggagaccgtcgaactgcgacagtatgacccagtggctgcactgttctttttcgacatcgacctgctgctgcagcgaggaccacagtacagcgagcaccctacttttacctcccagtatcggattcaggggaagctggagtacaggcacacctgggatcgccatgacgaaggagccgctcagggggacgatgacgtgtggacatctggcagtgattcagacgaggaactggtgacaactgagcgaaaaaccccccgggtgacaggaggaggggcaatggcaggggccagcaccagcgcagggcggaagcgaaaaagcgccagcagcgccacagcatgtaccgccggcgtgatgactagaggaaggctgaaggccgagtctacagtcgctcccgaggaagatactgacgaggatagtgacaatgaaatccacaaccccgccgtgttcacctggccaccttggcaggcagggattctggctcgcaacctggtccccatggtggcaaccgtccagggacagaatctgaagtatcaggagtttttctgggatgctaacgacatctaccggatttttgcagagctggaaggcgtgtggcagccagcagcccagcccaaacgacggagacatcgacaggacgctctgccaggaccttgtatcgccagcacaccaaagaagcacaggggc

(19) United States
(12) Patent Application Publication
(10) Pub. No.: US 2023/0022956 A1
(43) Pub. Date: Jan. 26, 2023

(54) IMMUNOTHERAPEUTIC COMPOSITIONS

(71) Applicants: GLAXOSMITHKLINE BIOLOGICALS SA, RIXENSART (BE); VARIATION BIOTECHNOLOGIES INC., OTTAWA (CA)

(72) Inventors: Kirsten SCHNEIDER-OHRUM, Rockville, MD (US); David Evander ANDERSON, Cambridge, MA (US)

(73) Assignees: GLAXOSMITHKLINE BIOLOGICALS SA, RIXENSART (BE); VARIATION BIOTECHNOLOGIES INC., OTTAWA (CA)

(21) Appl. No.: 17/940,117

(22) Filed: Sep. 8, 2022

Related U.S. Application Data
(62) Division of application No. 17/014,028, filed on Sep. 8, 2020.
(60) Provisional application No. 62/897,603, filed on Sep. 9, 2019.

(51) Int. Cl. A61K 39/245; A61K 9/127; A61K 39/21; A61K 39/39

(52) U.S. Cl. CPC A61K 39/245; A61K 9/127; A61K 39/21; A61K 39/39; A61K 2039/545

(57) ABSTRACT

The present disclosure provides compositions and methods useful for treating Glioblastoma Multiforme (GBM) which comprise virus-like particles (VLPs) comprising murine leukemia virus (MLV) core proteins and the human cytomegalovirus epitopes, gB and pp65, formulated with an adjuvant comprising a saponin and a TLR4 agonist.

### FIELD OF THE INVENTION

This invention is in the field of immune-oncology, in particular adjuvanted virus like particle vaccines of use in the treatment of human cytomegalovirus (HCMV) associated cancers such as glioblastoma multiforme (GBM).

### BACKGROUND

HCMV infection has been associated with a number of cancers including: GBMs (Cobbs 2002; Mitchel 2008), breast cancer (Taher 2013; Harkins 2010); prostate; medulloblastoma (Baryawno 2011; Libard 2014); meningioma (Libard 2014) and neuroblastoma (Wolmer-Solberg 2013).

GBM is the most common and aggressive primary form of brain tumour with median survival time being only three months without treatment. GBM affects 2 to 3 adults per 100,000 each year in the United States and Europe. In the United States alone each year, GBM is diagnosed in more than 20,000 people and is responsible for about 15,000 deaths.

There remains a need for alternative or improved therapeutic approaches for HCMV associated cancers, in particular GBM.

### SUMMARY

The present disclosure relates to compositions and methods useful for treatment of HCMV associated cancers including GBM. More particularly, the present disclosure provides adjuvanted compositions comprising virus like particles (VLPs) comprising a murine leukemia virus (MLV) gag protein and HCMV antigens gB and pp65, methods for their use and related aspects.

The present invention provides an immunogenic composition comprising:
(i) a virus like particle comprising:
   (a) a murine leukemia virus (MLV) gag protein;
   (b) an HCMV pp65 protein; and
   (c) an HCMV glycoprotein B (gB) protein;
and
(ii) an adjuvant comprising a saponin and a TLR4 agonist.

Suitably, the adjuvant comprises a saponin which is QS21 and a TLR4 agonist which is 3-de-O-acylated monophosphoryl lipid A (3D-MPL).

Also provided is a method for eliciting an immune response in a subject, said method comprising the administration of a saponin, a TLR4 agonist and a virus like particle comprising:
(a) a murine leukemia virus (MLV) gag protein;
(b) an HCMV pp65 protein; and
(c) an HCMV glycoprotein B (gB) protein.

Suitably, the saponin, TLR4 agonist and virus like particle are administered in the form of an immunogenic composition comprising the virus like particle and an adjuvant comprising the saponin and the TLR4 agonist.

Additionally provided is an immunogenic composition comprising:
(i) a virus like particle comprising:
   (a) a murine leukemia virus (MLV) gag protein;
   (b) an HCMV pp65 protein; and
   (c) an HCMV glycoprotein B (gB) protein;
and
   (ii) an adjuvant comprising a saponin and a TLR4 agonist;

for use in the treatment of an HCMV associated cancer.

The invention also provides a method for the treatment of an HCMV associated cancer in a subject, said method comprising the administration of an adjuvant comprising a saponin and a TLR4 agonist, and a virus like particle comprising:
(a) a murine leukemia virus (MLV) gag protein;
(b) an HCMV pp65 protein; and
(c) an HCMV glycoprotein B (gB) protein.

### LISTING OF SEQUENCES

The following is a list of sequences referred to herein:
SEQ ID NO: 1 is an MMLV-Gag Amino Acid Sequence
SEQ ID NO: 2 is MMLV-Gag Nucleotide Sequence
SEQ ID NO: 3 is a Codon Optimized MMLV-Gag Nucleotide Sequence
SEQ ID NO: 4 is a MMLV Gag—CMV pp65 Amino Acid Sequence
SEQ ID NO: 5 is a MMLV Gag—CMV pp65 Nucleotide Sequence
SEQ ID NO: 6 is a Codon Optimized MMLV Gag—CMV pp65 Nucleotide Sequence
SEQ ID NO: 7 is a Codon Optimized MMLV Gag—CMV pp65 Nucleotide Sequence
SEQ ID NO: 8 is a HCMV gB Amino Acid Sequence
SEQ ID NO: 9 is a HCMV gB Nucleotide Sequence
SEQ ID NO: 10 is a Codon Optimized HCMV gB Nucleotide Sequence
SEQ ID NO: 11 is a HCMV pp65 Amino Acid Sequence
SEQ ID NO: 12 is a HCMV pp65 Nucleotide Sequence
SEQ ID NO: 13 is a Codon Optimized HCMV pp65 Nucleotide Sequence

### DETAILED DESCRIPTION OF THE EMBODIMENTS

The present invention provides an immunogenic composition comprising:
(i) a virus like particle comprising:
   (a) a murine leukemia virus (MLV) gag protein;
   (b) an HCMV pp65 protein; and
   (c) an HCMV glycoprotein B (gB) protein;
and
(ii) an adjuvant comprising a saponin and a TLR4 agonist.

Suitably, the adjuvant comprises a saponin which is QS21 and a TLR4 agonist which is 3-de-O-acylated monophosphoryl lipid A (3D-MPL).

Also provided is a method for eliciting an immune response in a subject, said method comprising the administration of a saponin, a TLR4 agonist and a virus like particle comprising:
(a) a murine leukemia virus (MLV) gag protein;
(b) an HCMV pp65 protein; and
(c) an HCMV glycoprotein B (gB) protein.

Suitably, the saponin, TLR4 agonist and virus like particle are administered in the form of an immunogenic composition comprising the virus like particle and an adjuvant comprising the saponin and the TLR4 agonist.

The elicited immune response will typically comprise an antigen specific antibody and/or CD4+ T cell and/or CD8+ T cell response, suitably an antibody, CD4+ T cell and CD8+ T cell response, to one or both (suitably both) pp65 and gB proteins.

The invention also provides a method for the treatment of an HCMV associated cancer in a subject, said method comprising the administration of an adjuvant comprising a saponin and a TLR4 agonist, and a virus like particle comprising:
(a) a murine leukemia virus (MLV) gag protein;
(b) an HCMV pp65 protein; and
(c) an HCMV glycoprotein B (gB) protein.

The invention provides the use of a saponin, a TLR4 agonist and a virus like particle comprising:
(a) a murine leukemia virus (MLV) gag protein;
(b) an HCMV pp65 protein; and
(c) an HCMV glycoprotein B (gB) protein,

in the manufacture of a medicament, in particular in the manufacture of a medicament for the treatment of an HCMV associated cancer.

Additionally provided is an adjuvant comprising a saponin and a TLR4 agonist for use with a virus like particle comprising:
(a) a murine leukemia virus (MLV) gag protein;
(b) an HCMV pp65 protein; and
(c) an HCMV glycoprotein B (gB) protein.

Compositions comprising a VLP, a saponin and a TLR4 agonist may be provided as a kit of parts including the VLP, the saponin and the TLR4 agonist. For example, a first container may comprise the VLP and a second container comprise the saponin and the TLR4 agonist. Alternatively, a first container may comprise the VLP, a second container may comprise the saponin and a third container may comprise the TLR4 agonist.

While the components of the composition are conveniently administered as a single mixture to a particular administration site, components may be administered separately (or in incomplete combinations) either to the same or different sites (see for example WO2018114982 incorporated herein by reference for the purposes of providing information regarding the delivery of saponin and TLR4 agonist based adjuvants). If administered separately (or in incomplete combinations) either to the same or different sites, the administration is desirably sufficiently proximal in both administration time and administration location such that the outcome observed is substantially the same as would be observed through administration of a single mixture. Suitably separate administration, is within 2 hours, such as within 30 minutes, at locations suitably draining to the same lymph node, such as within 10 cm.

HCMV Associated Cancers

As mentioned previously, HCMV infection has been associated with a number of cancers (Nauclé r 2019) including: GBMs (Cobbs 2002; Mitchel 2008), breast cancer (Taher 2013; Harkins 2010); prostate; medulloblastoma (Baryawno 2011; Libard 2014); meningioma (Libard 2014) and neuroblastoma (Wolmer-Solberg 2013). HCMV, a β-herpesvirus, is a ubiquitously occurring pathogen. In an immunocompetent person, HCMV infection is normally unnoticed, having at most mild and nonspecific symptoms.

The term ‘HCMV associated cancer’ as used herein means a cancer wherein HCMV infection may be a contributory factor and wherein anti-HCMV treatment may be beneficial. Cancers associated with HCMV infection include breast, colon, ovarian and prostate cancer, rhabdomyosarcoma, hepatocellular cancer, salivary gland tumours, neuroblastoma and brain tumours (medulloblastoma and GBM). Consequently, in an embodiment of the invention the HCMV associated cancer is selected from breast, colon, ovarian and prostate cancer, rhabdomyosarcoma, hepatocellular cancer, salivary gland tumours, neuroblastoma and brain tumours (medulloblastoma and GBM). Of particular interest is GBM.

Subjects with GBM may be those experiencing their first occurrence or may be those experiencing a reoccurrence of GBM. In one embodiment the subject is one having their first occurrence of GBM. In a second embodiment the subject is one having a reoccurrence (such as the first reoccurrence) of GBM.

Suitably subjects will have small tumours, such as a maximum cross-section of 400 mm² or less based on two-dimensional magnetic resonance imaging. Typically, this will be at initiation of treatment.

A human subject desirably has a CD4/CD8 ratio of at least 2, such as at least 2.5 and in particular at least 3 at initiation of treatment. A human subject may have a CD4/CD8 ratio of less than 3, such as less than 2.5 and in particular less than 2 at initiation of treatment.

GBM responds poorly to treatment due to a number of factors including the localization of the tumour, the inherent resistance of the cells to chemotherapy, and brain cells' poor capacity for self-repair. Typically, GBM tumours are surgically removed to the extent possible, however, complete removal is usually impossible due to the rapid invasion of GBM cells into surrounding tissue. Radiation and chemotherapy are often used following surgical treatment in an attempt to delay progression of the disease. However, GBM tumours usually recur and median survival time in treated patients is only between twelve and fifteen months.

In recent years, immunotherapies have been proposed as treatments for GBM, based on the knowledge that T cells have been shown to kill tumour cells and infiltrate brain tumours. However, the development of immunotherapeutic agents to treat GBM has proven challenging because of the diversity of the tumour cells and the lack of a common tumour rejection antigen which could act as an immune target. In particular, GBM patients demonstrate a variety of different T-cell dysfunctions including anergy, tolerance and T-cell exhaustion (Woroniecka 2018). They also show a weakened antibody response. In one study, 31 percent of GBM patients with HCMV positive tumours lacked anti-HCMV antibodies (Rahbar 2015).

Immunotherapeutic approaches to treat GBM have been proposed based on the discovery of viral antigens in GBM tumour cells, which show lower expression in normal brain tissue. As early as 2002, it was discovered that human cytomegalovirus (HCMV) was present in GBM cells (Cobbs 2002). HCMV DNA and proteins are expressed in over 90% of GBM cells but not in the surrounding normal brain tissue (Dziurzynski 2012). Although the role of HCMV in GBM is not well understood, the HCMV glycoprotein B (gB) has been shown to mediate glioma cell entry by binding to the receptor tyrosine kinase PDGFR-alpha (PDGFRa), resulting in activation of the P13 kinase/Akt signaling pathway, which enhances both tumour cell growth and invasiveness (Cobbs 2014). Low levels of HCMV expression have been correlated with improved overall survival in GBM patients (Rahbar 2012).

The ubiquitous presence of HCMV in GBM cells has led to suggestions that HCMV antigens could constitute therapeutic targets for immunotherapeutic treatment. Of particular potential benefit to the use of HCMV antigens as targets is that they are recognized immunologically as being “foreign,” and T cells have a much higher affinity for foreign antigens than for self-antigens.

Some studies have investigated immunotherapy directed against HCMV antigens in the treatment of GBM. In one study, HCMV-specific T cells (CD4+and CD8+polyfunctional T cells) were shown to recognize and kill autologous GBM tumour cells, providing evidence that HCMV antigens are presented by tumour cells at immunologically relevant levels (Nair 2014). Extending these observations into the clinic, adoptive T cell therapy with autologous HCMV-specific T cells demonstrated encouraging early clinical results, with 4 out of 10 patients remaining disease free during the study period (Schuessler 2014).

While these preliminary studies showed promise for HCMV-targeted immunotherapies, other studies showed that GBM patients show a significantly lower immune response to HCMV compared to healthy persons (Liu 2018). In particular, GBM patients were shown to produce significantly lower anti-HCMV antibodies (IgG) compared to healthy subjects who are HCMV positive (Liu 2018). In one study, 31% of patients with GBM tumours that had HCMV completely lacked anti-CMV antibodies (Rahbar 2015). Accordingly, GBM patients have significant dysregulation of immunity against HCMV, which creates significant challenges in developing immunotherapeutic treatments based on HCMV antigens.

A need exists for an accessible immunotherapeutic treatment for GBM, which effectively targets GBM tumour cells but can be formulated for use in a broad patient population.

The present disclosure provides an immunotherapeutic composition and method of its use for treatment of HCMV associated cancer such as GBM. The immunogenic composition of the invention stimulates anti-HCMV T cell immunity against HCMV-expressing tumours, such as HCMV-expressing GBM tumours.

The immunotherapeutic composition comprises a virus-like particle (“VLP”). VLPs are multiprotein structures which are generally composed of one or more viral proteins. VLPs mimic the conformation of viruses but lack genetic material, and therefore are not infectious. They can form (or “self-assemble”) upon expression of a viral structural protein under appropriate circumstances. VLPs overcome some of the disadvantages of vaccines prepared using attenuated viruses because they can be produced without the need to have any live virus present during the production process. A wide variety of VLPs have been prepared. For example, VLPs including single or multiple capsid proteins either with or without envelope proteins and/or surface glycoproteins have been prepared. In some cases, VLPs are non-enveloped and assemble by expression of just one major capsid protein. In other cases, VLPs are enveloped and can comprise multiple antigenic proteins found in the corresponding native virus. Self-assembly of enveloped VLPs is more complex than non-enveloped VLPs because of the complex reactions required for fusion with the host cell membrane (Garrone 2011) and “budding” of the VLP to form a fully enveloped separate particle. Formation of intact VLPs can be confirmed by imaging of the particles using electron microscopy.

VLPs typically resemble their corresponding native virus and can be multivalent particulate structures. Presentation of surface glycoproteins in the context of a VLP is advantageous for induction of neutralizing antibodies against the polypeptide as compared to other forms of antigen presentation, e.g., soluble antigens not associated with a VLP. Neutralizing antibodies most often recognize tertiary or quaternary structures; this often requires presenting antigenic proteins, like envelope glycoproteins, in their native viral conformation. Antigens expressed on the surface of the VLPs also induce a CD4-restricted T helper cell response that can help elicit and sustain both neutralizing antibody and cytotoxic T lymphocyte (CTL) responses. In contrast, antigens expressed within the internal space of the VLP may promote CD8-restricted CTL responses through dendritic cell uptake of VLPs in a process referred to as cross-priming and presentation.

The VLPs of use in the present invention are typically derived from a retroviral vector. Retroviruses are enveloped RNA viruses that belong to the family Retroviridae. After infection of a host cell by a retrovirus, RNA is transcribed into DNA via the enzyme reverse transcriptase. DNA is then incorporated into the host cell's genome by an integrase enzyme and thereafter replicates as part of the host cell's DNA. The Retroviridae family includes the following genera Alpharetrovirus, Betaretrovirus, Gammearetrovirus, Deltaretrovirus, Epsilonretrovirus, Lentivirus and Spumavirus. The hosts for this family of retroviruses generally are vertebrates. Retroviruses produce an infectious virion containing a spherical nucleocapsid (the viral genome in complex with viral structural proteins) surrounded by a lipid bilayer derived from the host cell membrane.

Retroviral vectors can be used to generate VLPs that lack a retrovirus-derived genome and are therefore non-replicating. This is accomplished by replacement of most of the coding regions of the retrovirus with genes or nucleotide sequences to be transferred; so that the vector is incapable of making proteins required for additional rounds of replication. Depending on the properties of the glycoproteins present on the surface of the particles, VLPs have limited ability to bind to and enter the host cell but cannot propagate. Therefore, VLPs can be administered safely as an immunogenic composition.

The present invention utilizes VLPs comprising Murine Leukemia Virus (MLV) Gag protein and, in particular, a Moloney Murine Leukemia Virus (MMLV). Genomes of these retroviruses are readily available in databases.

The Gag proteins of retroviruses have an overall structural similarity and, within each group of retroviruses, are generally conserved at the amino acid level. Retroviral Gag proteins primarily function in viral assembly. Expression of Gag of some viruses (e.g., murine leukemia viruses, such as MMLV) in some host cells, can result in self-assembly of the expression product into

VLPs. The Gag gene expression product in the form of a polyprotein gives rise to the core structural proteins of the VLP. Functionally, the Gag polyprotein is divided into three domains: the membrane binding domain, which targets the Gag polyprotein to the cellular membrane, the interaction domain which promotes Gag polymerization and the late domain which facilitates release of nascent virions from the host cell. In general, the form of the Gag protein that mediates viral particle assembly is the polyprotein. Retroviruses assemble an immature capsid composed of the Gag polyprotein but devoid of other viral elements like viral protease with Gag as the structural protein of the immature virus particle.

The MMLV Gag gene encodes a 65kDa polyprotein precursor which is proteolytically cleaved into 4 structural proteins (Matrix (MA); p12; Capsid (CA); and Nucleocapsid (NC)), by MLV protease, in the mature virion. In the absence of MLV protease, the polyprotein remains uncleaved, and the resulting particle remains in an immature form. The gene encoding the MMLV nucleic acid is SEQ ID NO: 2. An exemplary codon optimized sequence of MMLV nucleic acid is provided as SEQ ID NO: 3.

Therefore, in some embodiments, a Gag polypeptide suitable for the present invention is substantially homologous to an MMLV Gag polypeptide which is SEQ ID NO:1. In some embodiments, a Gag polypeptide suitable for the present invention has an amino acid sequence at least 80%, ₈₅%_{, 90}%_{, 91}%_{, 92}%_{, 93}%_{, 94}%_{,} 95%, ₉₆%_{, 97}%_{, 98}0^{,}_{/0} , 99% or more homologous to SEQ ID NO:1. In some embodiments, a Gag polypeptide suitable for the present invention is substantially identical to, or identical to SEQ ID NO: 1.

In some embodiments, a suitable MMLV Gag polypeptide is encoded by a nucleic acid sequence having at least 80%, 85%, 90%, 91%, 92%, 93%, 94%, 95%, 96%, 97%, 98%, 99% sequence identity to SEQ ID NO:2. In some embodiments, a suitable MMLV Gag polypeptide is encoded by a nucleic acid sequence having SEQ ID NO: 2 or a codon degenerate version thereof.

As is well known to those of skill in the art, it is possible to improve the expression of a nucleic acid sequence in a host organism by replacing the nucleic acids coding for a particular amino acid (i.e. a codon) with another codon which is better expressed in the host organism. One reason that this effect arises is due to the fact that different organisms show preferences for different codons. The process of altering a nucleic acid sequence to achieve better expression based on codon preference is called codon optimization. Various methods are known in the art to analyze codon use bias in various organisms and many computer algorithms have been developed to implement these analyses in the design of codon optimized gene sequences. Therefore, in some embodiments, a suitable MMLV Gag polypeptide is encoded by a codon optimized version of a nucleic acid sequence encoding MMLV Gag and having at least 80%, 85%, 90%, 91%, 92%, 93%, 94%, 95%, 96%, 97%, 98%, 99% sequence identity to SEQ ID NO:3. In some embodiments, a suitable MMLV-Gag polypeptide is encoded by a nucleic acid sequence which is substantially identical to, or identical to, SEQ ID NO: 3.

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Examples of such programs are described in Altschul, et al., 1990, J. Mol. Biol., 215(3): 403-410; Altschul, et al., 1996, Methods in Enzymology 266:460-480; Altschul, et al., 1997 Nucleic Acids Res. 25:3389-3402; Baxevanis, et al., 1998, Bioinformatics: A Practical Guide to the Analysis of Genes and Proteins, Wiley; and Misener, et al., (eds.), 1999, Bioinformatics Methods and Protocols (Methods in Molecular Biology, Vol. 132), Humana Press. In addition to identifying homologous sequences, the programs mentioned above typically provide an indication of the degree of homology. In some embodiments, two sequences are considered to be substantially homologous if at least 50%, 55%, 60%, 65%, 70%, 75%, 80%, 85%, 90%, 91%, 92%, 93%, 94%, 95%, 96%, 97%, 98%, 99% or more of their corresponding residues are homologous over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

The Gag polypeptide used in the invention may be a modified retroviral Gag polypeptide containing one or more amino acid substitutions, deletions, and/or insertions as compared to a wild-type or naturally-occurring Gag polypeptide while retaining substantial self-assembly activity. Typically, in nature, a Gag protein includes a large C-terminal extension which may contain retroviral protease, reverse transcriptase, and integrase enzymatic activity. Assembly of VLPs, however, generally does not require the presence of such components. In some cases, a retroviral Gag protein alone (e.g., lacking a C-terminal extension, lacking one or more of genomic RNA, reverse transcriptase, viral protease, or envelope protein) can self-assemble to form VLPs both in vitro and in vivo (Sharma 1997).

In some embodiments, the VLP comprises a pp65 protein comprising an amino acid sequence which is at least 80%, 85%, 90%, 91%, 92%, 93%, 94%, 95%, 96%, 97%, 98%, 99% or more homologous to SEQ ID NO: 11. In some embodiments, the pp65 protein comprises a polypeptide encoded by a nucleic acid sequence at least 80%, 85%, 90%, 91%, 92%, 93%, 94%, 95%, 96%, 97%, 98%, 99% or more homologous to SEQ ID NO: 12.

The Gag polypeptide for use in accordance with the present invention suitably lacks a C-terminal extension and is typically expressed as a fusion protein with the pp65 antigen from HCMV. In naturally occurring HCMV, pp65 is located within the tegument between the capsid and the viral envelope. It is a major target of the cytotoxic T-cell response and is known to stimulate formation of T-helper cells and also induce cytotoxic T lymphocytes (CTL) against HCMV. The pp65 polypeptide is spliced in frame into the Gag polypeptide coding sequence, e.g., at the 3′ end of the Gag polypeptide coding sequence. The Gag polypeptide coding sequence and the pp65 antigen are expressed by a single promoter.

In some embodiments, the VLP comprises a fusion protein having an amino acid sequence which is at least 80%, 85%, 90%, 91%, 92%, 93%, 94%, 95%, 96%, 97%, 98%, 99% or more homologous to SEQ ID NO: 4. In some embodiments, the VLP comprises a fusion protein encoded by a nucleic acid sequence at least 80%, 85%, 90%, 91%, 92%, 93%, 94%, 95%, 96%, 97%, 98%, 99% or more homologous to SEQ ID NO: 5.

The VLPs of use in the invention also express an HCMV gB envelope glycoprotein on the surface of the VLP. gB is one of the major B-cell antigens in HCMV, inducing neutralizing, protective immune responses including potent humoral immune responses. The immunogenic composition of the present invention may comprise a VLP comprising a wild type envelope HCMV gB polypeptide, the sequence of which is SEQ ID NO: 8 or a codon degenerate version of SEQ ID NO. 8. A nucleic acid which encodes for the polypeptide is shown as SEQ ID NO: 9. A codon optimized version of SEQ ID NO: 9 is shown as SEQ ID NO: 10.

In some embodiments, the immunogenic composition of the invention comprises a VLP comprising a gB polypeptide comprising an amino acid sequence which is at least 80%, 85%, 90%, 91%, 92%, 93%, 94%, 95%, 96%, 97%, 98%, 99% or more homologous to SEQ ID NO: 8. In some embodiments, the polypeptide is encoded by a nucleic acid sequence at least 80%, 85%, 90%, 91%, 92%, 93%, 94%, 95%, 96%, 97%, 98%, 99% or more homologous to SEQ ID NO: 9. In some embodiments, the polypeptide is encoded by a codon optimized version of the nucleic acid sequence of SEQ ID NO: 9, which is at least 80%, 85%, 90%, 91%, 92%, 93%, 94%, 95%, 96%, 97%, 98%, 99% or more homologous to the SEQ ID NO: 10.

In some embodiments the VLP comprises a gB polypeptide consisting of an amino acid sequence which is at least 80%, 85%, 90%, 91%, 92%, 93%, 94%, 95%, 96%, 97%, 98%, 99% or more identical to SEQ ID NO: 8 and a Gag/pp65 fusion protein consisting of an amino acid sequence which is at least 80%, 85%, 90%, 91%, 92%, 93%, 94%, 95%, 96%, 97%, 98%, 99% or more identical to SEQ ID NO: 4.

It will be appreciated that a composition comprising VLPs will typically include a mixture of VLPs with a range of sizes. It is to be understood that the diameter values listed below correspond to the most frequent diameter within the mixture. In some embodiments >90% of the vesicles in a composition will have a diameter which lies within 50% of the most frequent value (e.g., 1000±500 nm). In some embodiments the distribution may be narrower, e.g., >90% of the vesicles in a composition may have a diameter which lies within 40, 30, 20, 10 or 5% of the most frequent value. In some embodiments, sonication or ultra-sonication may be used to facilitate VLP formation and/or to alter VLP size. In some embodiments, filtration, dialysis and/or centrifugation may be used to adjust the VLP size distribution.

In general, VLPs of the present disclosure may be of any size. In certain embodiments, the composition may include VLPs with diameters in the range of about 20 nm to about 300 nm. In some embodiments, a VLP is characterized in that it has a diameter within a range bounded by a lower limit of 20, 30, 40, 50, 60, 70, 80, 90, or 100 nm and bounded by an upper limit of 300, 290, 280, 270, 260, 250, 240, 230, 220, 210, 200, 190, 180, or 170 nm. In some embodiments, VLPs within a population show an average diameter within a range bounded by a lower limit of 20, 30, 40, 50, 60, 70, 80, 90, or 100 nm and bounded by an upper limit of 300, 290, 280, 270, 260, 250, 240, 230, 220, 210, 200, 190, 180, or 170 nm. In some embodiments, VLPs in a population have a polydispersity index that is less than 0.5 (e.g., less than 0.45, less than 0.4, or less than 0.3). In some embodiments, VLP diameter is determined by nanosizing. In some embodiments, VLP diameter is determined by electron microscopy.

VLPs in accordance with the present invention may be prepared according to general methodologies known to the skilled person. For example, nucleic acid molecules, reconstituted vectors or plasmids may be prepared using techniques well known to the skilled artisan. Recombinant expression of the polypeptides for VLPs requires construction of an expression vector containing a polynucleotide that encodes one or more polypeptide(s). Once a polynucleotide encoding one or more polypeptides has been obtained, the vector for production of the polypeptide may be produced by recombinant DNA technology using techniques known in the art. Expression vectors that may be utilized in accordance with the present invention include, but are not limited to mammalian and avian expression vectors, bacculovirus expression vectors, plant expression vectors (e.g., Cauliflower Mosaic Virus (CaMV), Tobacco Mosaic Virus (TMV)), plasmid expression vectors (e.g., Ti plasmid), among others.

The VLPs of the invention may be produced in any available protein expression system. Typically, the expression vector is transferred to a host cell by conventional techniques and the transfected cells are then cultured by conventional techniques to produce VLPs. In some embodiments, VLPs are produced using transient transfection of cells. In some embodiments, VLPs are produced using stably transfected cells. Typical cell lines that may be utilized for VLP production include, but are not limited to, mammalian cell lines such as human embryonic kidney (HEK) 293, WI 38, Chinese hamster ovary (CHO), monkey kidney (COS), HT1080, C10, HeLa, baby hamster kidney (BHK), 3T3, C127, CV-1, HaK, NS/O, and L-929 cells. Specific non-limiting examples include, but are not limited to, BALB/c mouse myeloma line

(NS0/1, ECACC No: 85110503); human retinoblasts (PER.C6 (CruCell, Leiden, The Netherlands)); monkey kidney CV1 line transformed by SV40 (COS-7, ATCC CRL 1651); human embryonic kidney line (293 cells subcloned for growth in suspension culture, Graham et al., J. Gen ViroL, 36:59 (1977)); baby hamster kidney cells (BHK, ATCC CCL 10); Chinese hamster ovary cells +/−DHFR (CHO, Urlaub and Chasin, Proc. Natl. Acad. Sci. USA, 77:4216 (1980)); mouse sertoli cells (TM4, Mather, Biol. Reprod., 23:243-251 (1980)); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumour (MMT 060562, ATCC CCL51); TRI cells (Mather et al., Annals N.Y. Acad. Sci., 383:44-68 (1982)); MRC 5 cells; FS4 cells; and a human hepatoma line (Hep G2). In some embodiments, cell lines that may be utilized for VLP production include insect (e.g., Sf-9, Sf-21, Tn-368, Hi5) or plant (e.g., Leguminosa, cereal, or tobacco) cells. It will be appreciated in some embodiments, particularly when glycosylation is important for protein function, mammalian cells are preferable for protein expression and/or VLP production (see, e.g., Roldao A et al., 2010 Expt Rev Vaccines 9:1149-76).

It will be appreciated that a cell strain may be chosen which modulates the expression of the inserted sequences, or modifies and processes the gene product in a specific way. Different cells have characteristic and specific mechanisms for post-translational processing and modification of proteins and gene products. Appropriate cell lines or host systems can be chosen to ensure the correct modification and processing of the foreign protein expressed. Generally, eukaryotic host cells (also referred to as packaging cells (e.g., 293T human embryo kidney cells)) which possess appropriate cellular machinery for proper processing of the primary transcript, glycosylation and phosphorylation of the gene product may be used in accordance with the present invention.

VLPs may be purified according to known techniques, such as centrifugation, gradients, sucrose-gradient ultracentrifugation, tangential flow filtration and chromatography (e.g., ion exchange (anion and cation), affinity and sizing column chromatography), or differential solubility, among others. Alternatively or additionally, cell supernatant may be used directly, with no purification step. Additional entities, such as additional antigens or adjuvants may be added to purified VLPs.

In order to produce the VLPs of the present disclosure, cells are co-transfected with two expression vectors, a first vector encoding a Gag-pp65 fusion polypeptide and a second vector encoding a gB envelope glycoprotein. The co-transfected HCMV gB plasmid enables particles budding from the cell surface to incorporate the gB protein into the lipid bilayer. As a result, “bivalent” VLPs comprising a HCMV pp65 non-structural protein and a HCMV gB envelope glycoprotein are produced.

The present inventors have previously reported development of HCMV VLP vaccines comprising a gB surface antigen presented in its native conformation which stimulated production of neutralizing antibodies, and a pp65 tegument protein which induced helper T cells (TH lymphocytes) and cytotoxic T cells (CTL) (see WO2013068847, which is incorporated herein by reference for the purposes of providing details on VLPs and their manufacture). In a study using peripheral blood mononuclear cells from healthy subjects, this VLP was shown to stimulate a CD4+and a CD8+ T cell immune response which was superior to the response generated by recombinant gB and pp65 antigens alone (see Example 3).

Saponins and TLR4 Agonists

The compositions, methods and uses of the present invention utilise a saponin and a TLR4 agonist.

Saponins A suitable saponin for use in the present invention is Quil A and its derivatives. Quil A is a saponin preparation isolated from the South American tree Quifiaja saponaria Molina and was first described as having adjuvant activity by Dalsgaard et al. in 1974 (“Saponin adjuvants”, Archiv. für die gesamte Virusforschung, Vol. 44, Springer Verlag, Berlin, p243-254). Purified fractions of Quil A have been isolated by HPLC which retain adjuvant activity without the toxicity associated with Quil A (see, for example, EP0362278). Fractions of general interest include QS7, QS17, QS18 and QS21, for example QS7 and QS21 (also known as QA7 and QA21). QS21 is a saponin of particular interest.

In certain embodiments of the present invention, the saponin is a derivative of Quillaja saponaria Molina Quil A, suitably an immunologically active fraction obtainable from Quil A, such as QS7, QS17, QS18 or QS21, in particular QS21.

Typically the saponin, such as Quil A and in particular QS21, is at least 90% pure, such as at least 95% pure, especially at least 98% pure, in particular 99% pure.

Purity of QS21 components may be determined by UV absorbance at 214nm as the proportion (e.g. at least 95%, especially at least 98%, in particular 99%) of QS21 components in the saponin used.

A beneficial feature of the present invention is that the saponin is presented in a less reactogenic composition where it is quenched with an exogenous sterol, such as cholesterol. In particular, QS21 is formulated with cholesterol-based liposomes as a delivery platform as further described herein. QS21 which is quenched with cholesterol shows equivalent immunostimulating properties to free QS21 but is less lytic and more stable (Garcon 2017).

TLR4 Agonists

A suitable example of a TLR4 agonist is a lipopolysaccharide, suitably a non-toxic derivative of lipid A, particularly a monophosphoryl lipid A and more particularly 3-de-O-acylated monophosphoryl lipid A (3D-MPL).

3D-MPL is sold under the name ‘MPL’ by GlaxoSmithKline Biologicals N.A. and is referred throughout the document as 3D-MPL. See, for example, US Pat. Nos. 4,436,727; 4,877,611; 4,866,034 and 4,912,094. 3D-MPL can be produced according to the methods described in GB 2 220 211 A. Chemically it is a mixture of 3-deacylated monophosphoryl lipid A with 4, 5 or 6 acylated chains. In the context of the present invention small particle 3D-MPL may be used to prepare the aqueous adjuvant composition. Small particle 3D-MPL has a particle size such that it may be sterile-filtered through a 0.22 um filter. Such preparations are described in WO94/21292. Suitably, powdered 3D-MPL is used to prepare aqueous adjuvant compositions of use in the present invention.

Other TLR4 agonists which can be used are aminoalkyl glucosaminide phosphates (AGPs) such as those described in WO98/50399 or US patent No. 6,303,347 (processes for preparation of AGPs are also described). Some AGPs are TLR4 agonists, and some are TLR4 antagonists.

Other TLR4 agonists which may be of use in the present invention include Glucopyranosyl Lipid Adjuvant (GLA) such as described in WO2008/153541 or WO2009/143457 or the literature articles Coler RN et al. (2011) Development and Characterization of Synthetic Glucopyranosyl Lipid Adjuvant System as a Vaccine Adjuvant. PLoS ONE 6(1): e16333. doi:10.1371/journal.pone.0016333 and Arias M A et al. (2012) Glucopyranosyl Lipid Adjuvant (GLA), a Synthetic TLR4 Agonist, Promotes Potent Systemic and Mucosal Responses to Intranasal Immunization with HIVgp140. PLoS ONE 7(7): e41144. doi:10.1371/journal.pone.0041144. WO2008/153541 or WO2009/143457 are incorporated herein by reference for the purpose of defining TLR4 agonists which may be of use in the present invention.

Typically the TLR4 agonist, such as the lipopolysaccharide and in particular 3D-MPL, is at least 90% pure, such as at least 95% pure, especially at least 98% pure, in particular 99% pure.

AGPs are TLR4 modulators. TLR4 recognizes bacterial LPS (lipopolysaccharide) and when activated initiates an innate immune response. AGPs are a monosaccharide mimetic of the lipid A portion of bacterial LPS and have been developed with ether and ester linkages on the “acyl chains” of the compound. Processes for making these compounds are known and disclosed, for example, in WO 2006/016997, U.S. Patent Nos. 7,288,640 and 6,113,918, and WO 01/90129, which are hereby incorporated by reference in their entireties for the purpose of defining AGPs of use in the present invention and their methods of manufacture. Other AGPs and related processes are disclosed in U.S. Patent No. 7,129,219, U.S. Patent No. 6,525,028 and U.S. Patent No 6,911,434, which are hereby incorporated by reference in their entireties for the purpose of defining AGPs of use in the present invention and their methods of manufacture. AGPs with ether linkages on the acyl chains employed in the composition of the invention are known and disclosed in WO 2006/016997 which is hereby incorporated by reference in its entirety for the purpose of defining AGPs of use in the present invention and their methods of manufacture. Of particular interest, are the AGPs set forth and described according to Formula (III) at paragraphs [0019] through [0021] in WO 2006/016997.

The term AGP therefore includes a compound of the formula:

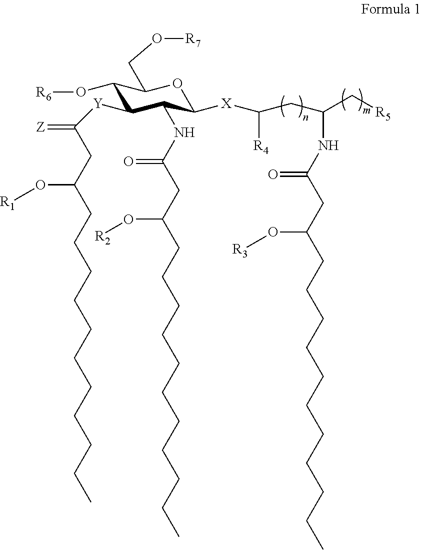

wherein

m is 0 to 6;

n is 0 to 4;

X is O or S, in particular O;

Y is O or NH;

Z is O or H;

each R₁, R₂, R₃ is selected independently from the group consisting of a C₁₋₂₉ acyl and a C₁₋₂₀ alkyl;

R₄ is H or methyl;

R₅ is selected independently from the group consisting of —H, —OH, —(C₁-C₄)alkoxy, —PO₃R₈R₉, —OPO₃R₈R₉, —SO₃R₈, —OSO₃R₈, —NR₈R₉, —SR₈, —CN, —NO₂, —CHO, —CO₂R₈, and —CONR₈R₉, wherein R₈ and R₉ are each independently selected from H and (C₁-C₄) alkyl; and each R₆ and R₇ is independently H or PO₃H₂.

The skilled person will appreciate that the AGP may be present in the form of a salt, particularly in the form of a pharmaceutically acceptable salt. Although non-pharmaceutically acceptable salts may be used during manufacture, they are desirably avoided.

The configuration of the 3′ stereogenic centres to which the normal fatty acyl residues (that is, the secondary acyloxy or alkoxy residues, e.g., R₁O, R₂O, and R₃O) are attached is R or S, suitably R (as designated by Cahn-Ingold-Prelog priority rules). Configuration of aglycon stereogenic centres to which R₄ and R₅ are attached can be R or S. All stereoisomers, both enantiomers and diastereomers, and mixtures thereof, are encompassed by the formula. The number of carbon atoms between heteroatom X and the aglycon nitrogen atom is determined by the variable “n”, which can be an integer from 0 to 4 (i.e. 0, 1, 2, 3 or 4), suitably an integer from 0 to 2 (i.e. 0, 1 or 2).

The chain length of normal fatty acids R₁, R₂, and R₃ will suitably be 6 to 20 carbons, especially 6 to 16 carbons, in particular 9 to 14 carbons. The chain lengths can be the same or different. Desirable embodiments include chain lengths where R₁, R₂ and R₃ are independently selected from 6, 8, 10, 12 or 14.

In one embodiment R₁, R₂ and R₃ are the same. In a second embodiment all of R₁, R₂ and R₃ are different. In a third embodiment one of R₁, R₂ and R₃ is different from the other two. The formula encompasses L/D-seryl, -threonyl, -cysteinyl ether and ester lipid AGPs, both agonists and antagonists and their homologs (n=1-4), as well as various carboxylic acid bioisosteres (i.e. R₅ is an acidic group capable of salt formation; the phosphate can be either on 4- or 6- position of the glucosamine unit, but suitably is in the 4-position).

In a particular embodiment of the AGP compound, n is 0, R₅ is CO₂H, R₆ is PO₃H₂, and R₇ is H. Such AGP compounds are therefore defined by Formula 1 a:

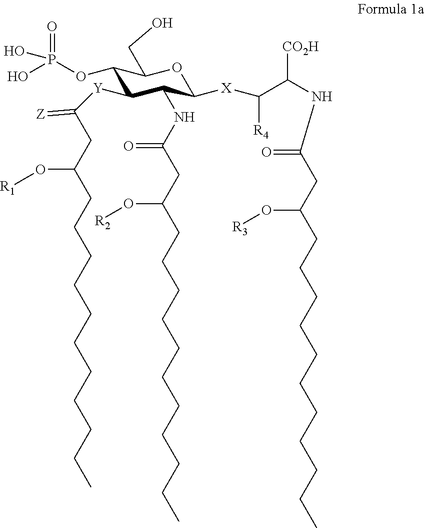

wherein X is O or S; Y is 0 or NH; Z is O or H; each R₁, R₂, R₃ is selected independently from the group consisting of a C₁₋₂₀ acyl and a C₁₋₂₀ alkyl; and R₄ is H or methyl.

In Formula la the configuration of the 3′ stereogenic centres to which the normal fatty acyl residues (that is, the secondary acyloxy or alkoxy residues, e.g., R₁O, R₂O, and R₃O) are attached as R or S, preferably R (as designated by Cahn-Ingold-Prelog priority rules). Configuration of aglycon stereogenic centres to which R₄ and CO₂H are attached can be R or S. All stereoisomers, both enantiomers and diastereomers, and mixtures thereof are encompassed by the formula.

Formula la encompasses L/D-seryl, -threonyl, -cysteinyl ether or ester lipid AGPs, both agonists and antagonists.

In both Formula 1 and Formula 1 a, Z is O attached by a double bond or two hydrogen atoms which are each attached by a single bond. That is, the compound is ester-linked when Z═Y═O; amide-linked when Z═O and Y═NH; and ether-linked when Z═H/H and Y═O.

A particular compound of Formula 1 is referred to as CRX601:

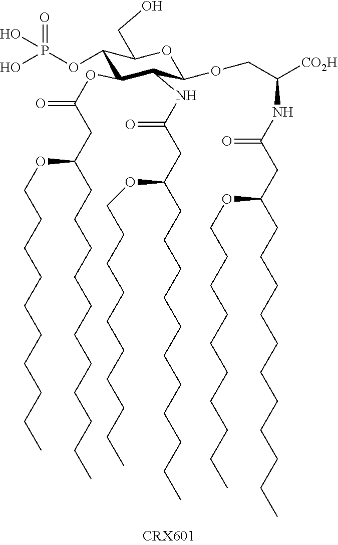

Glucopyranosyl Lipid Adjuvant

GLA are TLR4 modulators such as described in WO2008/153541 or WO2009/143457 or the literature articles Coler RN et al. (2011) Development and Characterization of Synthetic Glucopyranosyl Lipid Adjuvant System as a Vaccine Adjuvant. PLoS ONE 6(1): e16333. doi:10.1371/journal.pone.0016333 and Arias MA et al. (2012) Glucopyranosyl Lipid Adjuvant (GLA), a Synthetic TLR4 Agonist, Promotes Potent Systemic and Mucosal Responses to Intranasal Immunization with HIVgp140. PLoS ONE 7(7): e41144. doi:10.1371/journal.pone.0041144.

The term GLA therefore includes a compound of the formula:

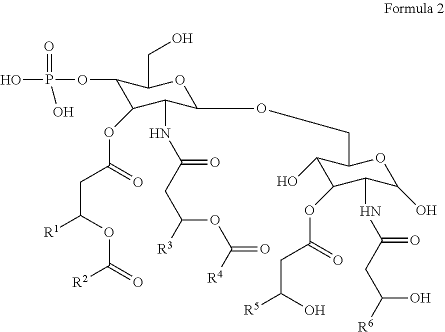

wherein:

R¹, R³, R⁵ and R⁶ are each independently C₁₁₋₂₀alkyl; and

R² and R⁴ are each independently C₁₂₋₂₀alkyl; and salts, such as pharmaceutically acceptable salts, thereof.

Of particular interest are compounds of Formula 2 having the stereochemistry:

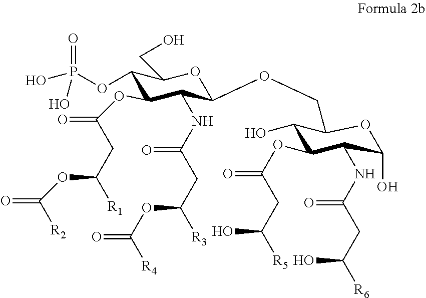

and salts, such as pharmaceutically acceptable salts, thereof.

Suitably, for compounds of Formula 2 and 2b, all of R¹ to R⁶ are linear alkyl groups.

Suitably each of R¹, R³, R⁵ and R⁶ is independently selected from C₁₁₋₁₅alkyl. Suitably each of R¹, R³, R⁵ and R⁶ is identical. Desirably, each of R¹, R³, R⁵ and R⁶ is C₁₁alkyl.

Suitably each of R² and R⁴ is independently selected from C₁₂₋₁₆alkyl. Suitably each of R² and R⁴ is identical. Suitably each of R² and R⁴ is identical. Desirably, each of R² and R⁴ is C₁₃alkyl.

TLR4 agonists of interest include:

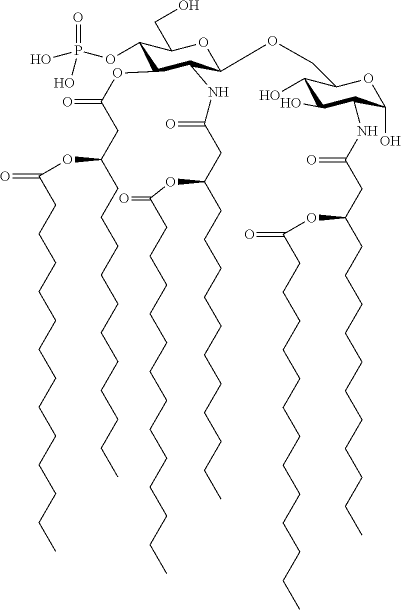

3-deacyl monophosphoryl hexa-acyl lipid A.

Another TLR4 agonist of interest is:

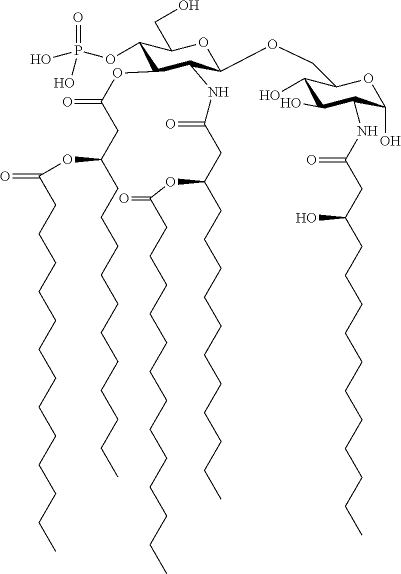

3-deacyl monophosphoryl lipid A.

A TLR agonist of interest is dLOS (as described in Han, 2014):

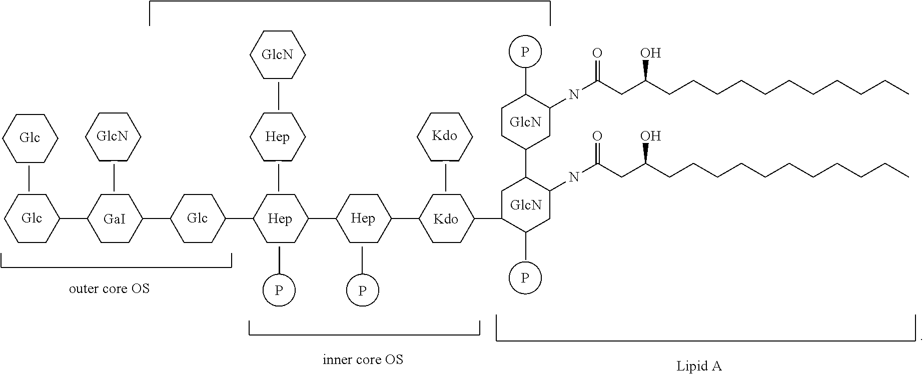

In one embodiment the TLR4 agonist is in the form of a salt, such as pharmaceutically acceptable salt. In a second embodiment the TLR4 agonist is not in the form of a salt.

Typically GLA, is at least 90% pure, such as at least 95% pure, especially at least 98% pure, in particular 99% pure.

The subject is typically a mammal and in particular is a human. The human may be a human child. For example, at the time of administration of the first dose, a human subject may be less than 18 years old. The human may be a human adult. For example, at the time of administration of the first dose, a human subject may be 18 to 60 years old, such as 18 to 40 years old.

The human may be an elderly adult. For example, at the time of administration of the first dose, a human subject may be greater than 60 years old (e.g. 60 to 80 years old), such as greater than 65 years old. The human may be 18-70 years of age.

A typical human dose comprises VLPs having 1 to 25 ug of pp65 protein content, such as 5 to 20 ug, in particular 8 to 12 ug, especially 10 ug. A typical human dose comprises VLPs having a gB protein content of 1/200^{th} to 1/10^{th} of content of pp65, such as 1/120^{th} to 1/40^{th} of content of pp65, in particular 1/100th to 1/60th of content of pp65 on a weight basis. Protein content can be determined by methods such as quantitative ELISA. When the pp65 protein is present in a fusion protein, pp65 content is based on the pp65 portion of the fusion protein.

Administration will typically be repeated as necessary. For example, administration may be repeated every week to every 6 months, such as every 2 weeks to every 6 weeks, for example around every 4 weeks (+/−3 days).

Compositions may comprise at least one additional pharmaceutically acceptable excipient(s), adjuvant(s) and/or carrier(s). Typical carriers include emulsions, ISCOMS and liposomes, particularly liposomes.

The term liposome' is well known in the art and defines a general category of vesicles which comprise one or more lipid bilayers surrounding an aqueous space. Liposomes thus consist of one or more lipid and/or phospholipid bilayers and can contain other molecules, such as proteins or carbohydrates, in their structure. Because both lipid and aqueous phases are present, liposomes can encapsulate or entrap water-soluble material, lipid-soluble material, and/or amphiphilic compounds.

Liposome size may vary from 30 nm to several um depending on the phospholipid composition and the method used for their preparation. In the present invention, the liposome size will be in the range of 50 nm to 200 nm, especially 60 nm to 180 nm, such as 70-165 nm. Optimally, the liposomes should be stable and have a diameter of -100 nm to allow convenient sterilization by filtration.

Structural integrity of the liposomes may be assessed by methods such as dynamic light scattering (DLS) measuring the size (Z-average diameter, Zav) and polydispersity of the liposomes, or, by electron microscopy for analysis of the structure of the liposomes. Suitably the average particle size is between 95 and 120 nm, and/or, the polydispersity (Pdl) index is not more than 0.35, in particular not more than 0.3, such as not more than 0.25. In one embodiment the average particle size is between 95 and 120 nm, and/or, the polydispersity (Pdl) index is not more than 0.2.

The liposomes of the present invention may contain phosphatidylcholine lipid, such as dioleoyl phosphatidylcholine (DOPC). Suitably the liposomes also contain a sterol, such as cholesterol.

The saponin, such as QS21, can be used at amounts between 1 and 100 ug per human dose such as 20 to 60 ug. QS21 may be used at a level of about 50 ug. Examples of suitable ranges are 40-60 ug, suitably 45-55 ug or 49-51 ug, such as 50 ug. In a further embodiment, the human dose comprises QS21 at a level of about 25 ug. Examples of lower ranges include 20-30 ug, suitably 22-28 ug or 24-26 ug, such as 25 ug.

The TLR4 agonist, such as 3D-MPL, can be used at amounts between 1 and 100 ug per human dose, such as 20 to 60 ug. 3D-MPL may be used at a level of about 50 ug. Examples of suitable ranges are 40-60 ug, suitably 45-55 ug or 49-51 ug, such as 50 ug. In a further embodiment, the human dose comprises 3D-MPL at a level of about 25 ug. Examples of lower ranges include 20-30 ug, suitably 22-28 ug or 24-26 ug, such as 25 ug.

The weight ratio of TLR4 agonist to saponin is suitably between 1:5 to 5:1, especially 1.2:1 to 1:1.2, such as 1:1.

Human doses intended for children may be reduced compared to those intended for an adult (e.g. reduction by 50%).

The ratio of saponin:DOPC will typically be in the order of 1:50 to 1:10 (w/w), suitably between 1:25 to 1:15 (w/w), and preferably 1:22 to 1:18 (w/w), such as 1:20 (w/w).

In a further embodiment, a buffer is added to the composition. The pH of a liquid preparation is adjusted in view of the components of the composition and necessary suitability for administration to the subject. Suitably, the pH of a liquid mixture is at least 4, at least 5, at least 5.5, at least 5.8, at least 6. The pH of the liquid mixture may be less than 9, less than 8, less than 7.5 or less than 7. In other embodiments, pH of the liquid mixture is between 4 and 9, between 5 and 8, such as between 5.5 and 8. Consequently, the pH will suitably be between 6-9, such as 6.5-8.5. In a particularly preferred embodiment the pH is between 5.8 and 6.4.

An appropriate buffer may be selected from acetate, citrate, histidine, maleate, phosphate, succinate, tartrate and TRIS. In one embodiment, the buffer is a phosphate buffer such as Na/Na₂PO₄, Na/K₂PO₄ or K/K₂PO₄.

The buffer can be present in the liquid mixture in an amount of at least 6mM, at least 10 mM or at least 40mM. The buffer can be present in the liquid mixture in an amount of less than 100 mM, less than 60 mM or less than 40 mM.

It is well known that for parenteral administration solutions should have a pharmaceutically acceptable osmolality to avoid cell distortion or lysis. A pharmaceutically acceptable osmolality will generally mean that solutions will have an osmolality which is approximately isotonic or mildly hypertonic. Suitably the compositions of the present invention when reconstituted will have an osmolality in the range of 250 to 750 mOsm/kg, for example, the osmolality may be in the range of 250 to 550 mOsm/kg, such as in the range of 280 to 500 mOsm/kg. In a particularly preferred embodiment the osmolality may be in the range of 280 to 310 mOsm/kg.

Osmolality may be measured according to techniques known in the art, such as by the use of a commercially available osmometer, for example the AdvancedTM Model 2020 available from Advanced Instruments Inc. (USA).

An “isotonicity agent” is a compound that is physiologically tolerated and imparts a suitable tonicity to a formulation to prevent the net flow of water across cell membranes that are in contact with the formulation. In some embodiments, the isotonicity agent used for the composition is a salt (or mixtures of salts), conveniently the salt is sodium chloride, suitably at a concentration of approximately 150 nM. In other embodiments, however, the composition comprises a non-ionic isotonicity agent and the concentration of sodium chloride in the composition is less than 100 mM, such as less than 80 mM, e.g. less than 50 mM, such as less 40 mM, less than 30 mM and especially less than 20 mM. The ionic strength in the composition may be less than 100 mM, such as less than 80 mM, e.g. less than 50 mM, such as less 40 mM or less than 30 mM.

In a particular embodiment, the non-ionic isotonicity agent is a polyol, such as sucrose and/or sorbitol. The concentration of sorbitol may e.g. between about 3% and about 15% (w/v), such as between about 4% and about 10% (w/v). Adjuvants comprising an immunologically active saponin fraction and a TLR4 agonist wherein the isotonicity agent is salt or a polyol have been described in WO2012/080369.

Suitably, a human dose volume is between 0.05 ml and 1 ml, such as between 0.1 and 0.5 ml, in particular a dose volume of about 0.5 ml, or 0.7 ml. The volumes of the compositions used may depend on the delivery route and location, with smaller doses being given by the intradermal route. A unit dose container may contain an overage to allow for proper manipulation of materials during administration of the unit dose.

Such compositions may optionally be administered in combination with one or more additional therapeutically active substances such as chemotherapeutic treatments.

Compositions are intended for administration and therefore will typically be provided in a sterile injectable form (e.g., a form that is suitable for intramuscular injection). For example, in some embodiments, pharmaceutical compositions are provided in a liquid (e.g. aqueous) dosage form that is suitable for injection.

In some embodiments, provided compositions comprise one or more pharmaceutically acceptable excipients (e.g., preservative, inert diluent, dispersing agent, surface active agent and/or emulsifier, buffering agent, etc.). Suitable excipients include, for example, water, saline, dextrose, sucrose, trehalose, glycerol, ethanol, or similar, and combinations thereof. Remington's The Science and Practice of Pharmacy, 21^{st} Edition, A. R. Gennaro, (Lippincott, Williams & Wilkins, Baltimore, Md., 2006) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the composition, its use is contemplated to be within the scope of this invention. In some embodiments, compositions comprise one or more preservatives. In some embodiments, compositions comprise no preservative.

In some embodiments, compositions are provided in a form that can be refrigerated and/or frozen. In some embodiments, reconstituted solutions and/or liquid dosage forms may be stored for a certain period of time after reconstitution (e.g., 2 hours, 12 hours, 24 hours, 2 days, 5 days, 7 days, 10 days, 2 weeks, a month, two months, or longer). In some embodiments, storage of VLP formulations for longer than the specified time results in VLP degradation.

Formulations of the compositions described herein may be prepared by any method known or hereafter developed in the art of pharmacology. In some embodiments, such preparatory methods include the step of bringing active ingredient into association with one or more excipients and/or one or more other accessory ingredients, and then, if necessary and/or desirable, packaging the product into a desired single- or multi-dose unit.

A composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to a dose which would be administered to a subject and/or a convenient fraction of such a dose such as, for example, one-half or one-third of such a dose.

Relative amounts of active ingredient, pharmaceutically acceptable excipient, and/or any additional ingredients in a pharmaceutical composition in accordance with the invention may vary, depending upon the identity, size, and/or condition of the subject and/or depending upon the route by which the composition is to be administered.

In some embodiments, treatment includes multiple administrations, appropriately spaced in time, of the composition of the present disclosure. Compositions described herein will generally be administered for such a time as they continue to induce an immune response, or until such time as the patient experiences progression of their disease. In a preferred embodiment of the invention, the composition of the invention is administered every four weeks.

The preferred dosage may vary from subject to subject and may depend on several factors. Thus, it will be appreciated that, in general, the precise dose used will be as determined by the prescribing physician and will depend not only on the weight of the subject, but also on the age of the subject and, possibly, the progression of the disease and the degree of immune dysregulation against HCMV in the patient.

In certain embodiments, provided compositions may be formulated for delivery parenterally, e.g., by injection. In such embodiments, administration may be, for example, intravenous, intramuscular, intradermal, or subcutaneous, or via by infusion or needleless injection techniques. In a preferred embodiment, the compositions are formulated for intramuscular injection.

Administration of compositions according to the present invention may improve quality of life of the subject, alleviate symptoms, slow progression and/or extend survival.

The present invention is further illustrated by the following clauses: Clause 1. An immunogenic composition comprising:
(i) a virus like particle (VLP) comprising:
   (a) a murine leukemia virus (MLV) gag protein;
   (b) an HCMV pp65 protein; and
   (c) an HCMV glycoprotein B (gB) protein;
and
(ii) an adjuvant comprising a saponin and a TLR4 agonist.

Clause 2. The immunogenic composition according to clause 1, for use in the treatment of an HCMV associated cancer.

Clause 3. A kit for the preparation of an immunogenic composition, said kit comprising:
(i) a first container comprising a virus like particle comprising:
   (a) a murine leukemia virus (MLV) gag protein;
   (b) an HCMV pp65 protein; and
   (c) an HCMV glycoprotein B (gB) protein;
and
(ii) a second container comprising an adjuvant comprising a saponin and a TLR4 agonist.

Clause 4. A kit for the preparation of an immunogenic composition, said kit comprising:
(i) a first container comprising a virus like particle comprising:
   (a) a murine leukemia virus (MLV) gag protein;
   (b) an HCMV pp65 protein; and
   (c) an HCMV glycoprotein B (gB) protein;
(ii) a second container comprising a saponin;
(iii) a third container comprising a TLR4 agonist.

Clause 5. A saponin for use with
(i) a virus like particle comprising:
   (a) a murine leukemia virus (MLV) gag protein;
   (b) an HCMV pp65 protein; and
   (c) an HCMV glycoprotein B (gB) protein;
and
(ii) a TLR4 agonist;

to elicit an immune response in a subject.

Clause 6. A TLR4 agonist for use with
(i) a virus like particle comprising:
   (a) a murine leukemia virus (MLV) gag protein;
   (b) an HCMV pp65 protein; and
   (c) an HCMV glycoprotein B (gB) protein;
and
(ii) a saponin;

to elicit an immune response in a subject.

Clause 7. An adjuvant comprising a saponin and a TLR4 agonist for use with a virus like particle comprising:
(a) a murine leukemia virus (MLV) gag protein;
(b) an HCMV pp65 protein; and
(c) an HCMV glycoprotein B (gB) protein.

Clause 8. A method for eliciting an immune response in a subject, said method comprising the administration of a saponin, a TLR4 agonist and a virus like particle comprising:
(a) a murine leukemia virus (MLV) gag protein;
(b) an HCMV pp65 protein; and
(c) an HCMV glycoprotein B (gB) protein.

Clause 9. The method according to clause 8, wherein the saponin, the TLR4 agonist and the virus like particle are administered in the form of an immunogenic composition according to clause 1.

Clause 10. A method for the treatment of an HCMV associated cancer in a subject, said method comprising the administration of a saponin, a TLR4 agonist and a virus like particle comprising:
(a) a murine leukemia virus (MLV) gag protein;
(b) an HCMV pp65 protein; and
(c) an HCMV glycoprotein B (gB) protein.

Clause 11. Use of a saponin, a TLR4 agonist and a virus like particle comprising:
(a) a murine leukemia virus (MLV) gag protein;
(b) an HCMV pp65 protein; and
(c) an HCMV glycoprotein B (gB) protein,

in the manufacture of a medicament, in particular in the manufacture of a medicament for the treatment of an HCMV associated cancer.

Clause 12. The composition, kit, saponin, TLR4 agonist, adjuvant, method or use according to any one of clauses 1 to 11, wherein the MLV Gag protein is a Moloney murine leukemia virus (MMLV) Gag protein.

Clause 13. The composition, kit, saponin, TLR4 agonist, adjuvant, method or use according to any one of clauses 1 to 12, wherein the VLP comprises a MLV Gag polypeptide comprising, such as consisting of, an amino acid sequence at least 80%, such as at least 90%, in particular at least 95%, especially at least 98% identical to SEQ ID NO:1.

Clause 14. The composition, kit, saponin, TLR4 agonist, adjuvant, method or use according to any one of clauses 1 to 13, wherein the VLP comprises a polypeptide encoded by a nucleic acid sequence having at least 80%, such as at least 90%, in particular at least 95%, especially at least 98% identical to SEQ ID NO:2.

Clause 15. The composition, kit, saponin, TLR4 agonist, adjuvant, method or use according to any one of clauses 1 to 14, wherein the VLP comprises a gB protein comprising, such as consisting of, an amino acid sequence which is at least 80%, such as at least 90%, in particular at least 95%, especially at least 98% identical to SEQ ID NO: 8.

Clause 16. The composition, kit, saponin, TLR4 agonist, adjuvant, method or use according to any one of clauses 1 to 15, wherein the VLP comprises a gB protein encoded by a nucleic acid sequence having at least 80%, such as at least 90%, in particular at least 95%, especially at least 98% identical to SEQ ID NO:9.

Clause 17. The composition, kit, saponin, TLR4 agonist, adjuvant, method or use according to any one of clauses 1 to 16, wherein the VLP comprises a pp65 protein comprising, such as consisting of, an amino acid sequence which is at least 80%, such as at least 90%, in particular at least 95%, especially at least 98% identical to SEQ ID NO: 11.

Clause 18. The composition, kit, saponin, TLR4 agonist, adjuvant, method or use according to any one of clauses 1 to 17, wherein the VLP comprises pp65 protein encoded by a nucleic acid sequence having at least 80%, such as at least 90%, in particular at least 95%, especially at least 98% identical to SEQ ID NO:12.

Clause 19. The composition, kit, saponin, TLR4 agonist, adjuvant, method or use according to any one of clauses 1 to 18, wherein the VLP comprises a fusion of the MLV Gag and HCMV pp65 protein.

Clause 20. The composition, kit, saponin, TLR4 agonist, adjuvant, method or use according to clause 19, wherein the MLV Gag is N-terminally fused, directly or indirectly, to the pp65 protein.

Clause 21. The composition, kit, saponin, TLR4 agonist, adjuvant, method or use according to clause 20, wherein the VLP comprises a fusion protein comprising, such as consisting of, an amino acid sequence at least 80%, such as at least 90%, in particular at least 95%, especially at least 98% identical to SEQ ID NO:4.

Clause 22. The composition, kit, saponin, TLR4 agonist, adjuvant, method or use according to any one of clauses 1 to 21, wherein the saponin is Quil A or a derivative thereof.

Clause 23. The composition, kit, saponin, TLR4 agonist, adjuvant, method or use according to clause 22, wherein the saponin is QS21.

Clause 24. The composition, kit, saponin, TLR4 agonist, adjuvant, method or use according to any one of clauses 1 to 23, wherein the TLR4 agonist is a lipopolysaccharide.

Clause 25. The composition, kit, saponin, TLR4 agonist, adjuvant, method or use according to clause 24, wherein the lipopolysaccharide is a monophosphoryl lipid A.

Clause 26. The composition, kit, saponin, TLR4 agonist, adjuvant, method or use according to clause 25, wherein the monophosphoryl lipid A is 3-de-O-acylated monophosphoryl lipid A (3D-MPL).

Clause 27 The composition, kit, saponin, TLR4 agonist, adjuvant, method or use according to any one of clauses 1 to 24, wherein the TLR4 agonist is an AGP, such as a compound of Formula 1 or Formula 1 a, in particular CRX601.

Clause 28. The composition, kit, saponin, TLR4 agonist, adjuvant, method or use according to any one of clauses 1 to 24, wherein the TLR4 agonist is a glucopyranosyl lipid adjuvant, such as a compound of Formula 2 or Formula 2b, in particular:

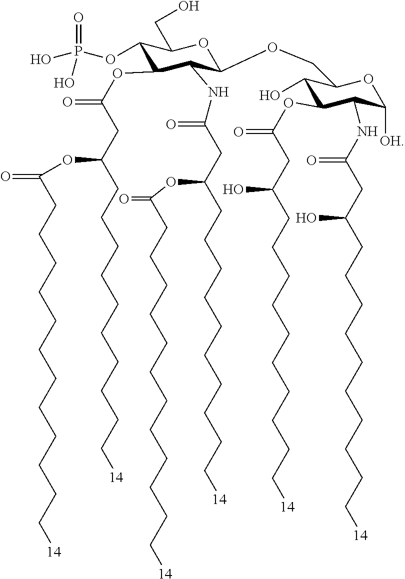

Clause 29. The composition, kit, saponin, TLR4 agonist, adjuvant, method or use according to any one of clauses 1 to 24, wherein the TLR4 agonist is:

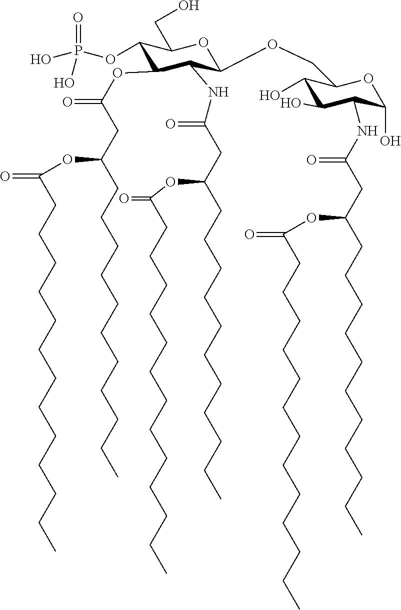

3-deacyl monophosphoryl hexa-acyl lipid A.

Clause 30. The composition, kit, saponin, TLR4 agonist, adjuvant, method or use according to any one of clauses 1 to 24, wherein the TLR4 agonist is:

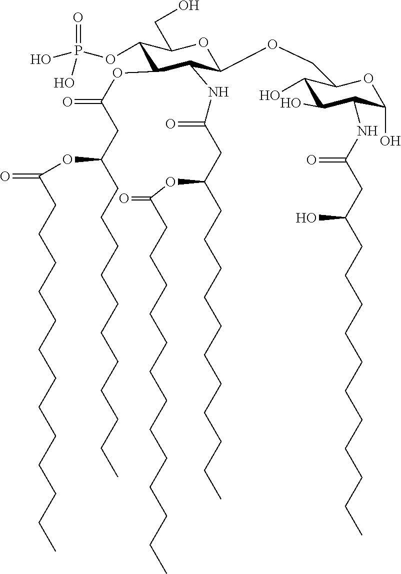

3-deacyl monophosphoryl lipid A.

Clause 31. The composition, kit, saponin, TLR4 agonist, adjuvant, method or use according to any one of clauses 1 to 24, wherein the TLR4 agonist is:

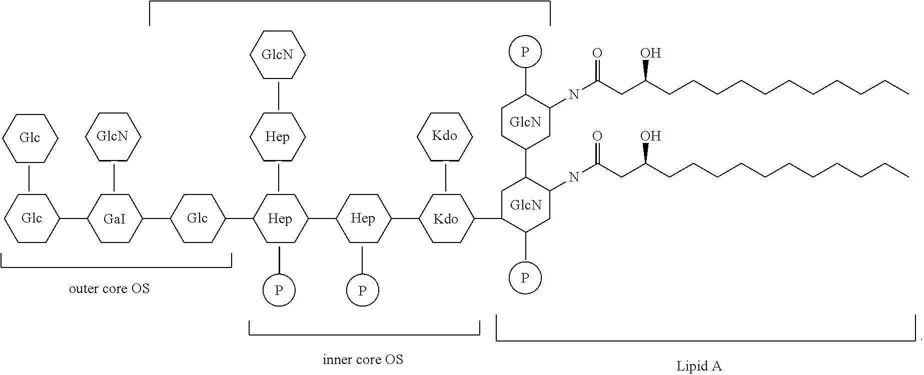

Clause 32. The composition, kit, saponin, TLR4 agonist, adjuvant, method or use according to any one of clauses 1 to 31, for administration to a human, such as a human aged 18 to 70.

Clause 33. The composition, kit, saponin, TLR4 agonist, adjuvant, method or use according to any one of clauses 1 to 32, for use in the treatment of an HCMV associated cancer selected from breast, colon, ovarian and prostate cancer, rhabdomyosarcoma, hepatocellular cancer, salivary gland tumours, neuroblastoma and brain tumours (such as medulloblastoma and GBM).

Clause 34. The composition, kit, saponin, TLR4 agonist, adjuvant, method or use according to any one of clauses 1 to 33, for use in the treatment of GBM.

Clause 35. The composition, kit, saponin, TLR4 agonist, adjuvant, method or use according to clause 34, for use in the treatment of GBM in a subject experiencing their first occurrence.

Clause 36. The composition, kit, saponin, TLR4 agonist, adjuvant, method or use according to clause 34, for use in the treatment of GBM in a subject experiencing a reoccurrence, such as the first reoccurrence.

Clause 37. The composition, kit, saponin, TLR4 agonist, adjuvant, method or use according to any one of clauses 34 to 36, for use in the treatment of GBM in a subject having a tumour with a maximum cross-sectional area of 400 mm² or less.

Clause 38. The composition, kit, saponin, TLR4 agonist, adjuvant, method or use according to any one of clauses 1 to 37, for use in a subject having a CD4/CD8 ratio of at least 2, such as at least 2.5, at initiation of treatment.

Clause 39. The composition, kit, saponin, TLR4 agonist, adjuvant, method or use according to clause 38, for use in a subject having a CD4/CD8 ratio of at least 3 at initiation of treatment.

Clause 40. The composition, kit, saponin, TLR4 agonist, adjuvant, method or use according to any one of clauses 1 to 37, for use in a subject having a CD4/CD8 ratio of less than 3, such as less than 2.5 and in particular less than 2 at initiation of treatment.

Clause 41. The composition, kit, saponin, TLR4 agonist, adjuvant, method or use according to any one of clauses 1 to 40, for use in eliciting an antigen specific antibody response.

Clause 42. The composition, kit, saponin, TLR4 agonist, adjuvant, method or use according to any one of clauses 1 to 41, for use in eliciting an antigen specific CD4+ T cell response.

Clause 43. The composition, kit, saponin, TLR4 agonist, adjuvant, method or use according to any one of clauses 1 to 42, for use in eliciting an antigen specific CD8+ T cell response.

Clause 44. The composition, kit, saponin, TLR4 agonist, adjuvant, method or use according to any one of clauses 1 to 43, wherein the antigen specific response is to pp65.

Clause 45. The composition, kit, saponin, TLR4 agonist, adjuvant, method or use according to any one of clauses 1 to 44, wherein the antigen specific response is to gB.

Clause 46. The composition, kit, saponin, TLR4 agonist, adjuvant, method or use according to any one of clauses 1 to 45, wherein administration is intramuscularly.

Clause 47. The composition, kit, saponin, TLR4 agonist, adjuvant, method or use according to any one of clauses 1 to 46, wherein a human dose comprises 1 to 25 ug of pp65 protein content, such as 5 to 20 ug, in particular 8 to 12 ug, especially 10 ug.

Clause 48. The composition, kit, saponin, TLR4 agonist, adjuvant, method or use according to any one of clauses 1 to 47, wherein a human dose comprises a gB protein content of 1/200^{th} to 1/10^{th} of content of pp65, such as 1/120^{th} to 1/40^{th} of content of pp65, in particular 1/100^{th} to 1/60^{th} of content of pp65 on a weight basis.

Clause 49. The composition, kit, saponin, TLR4 agonist, adjuvant, method or use according to any one of clauses 1 to 48, wherein a human dose comprises 1 to 100 ug saponin, such as 50 ug.

Clause 50. The composition, kit, saponin, TLR4 agonist, adjuvant, method or use according to any one of clauses 1 to 49, wherein a human dose comprises 1 to 100 ug TLR4 agonist, such as 50 ug.

Clause 51. The composition, kit, saponin, TLR4 agonist, adjuvant, method or use according to any one of clauses 1 to 50, wherein the saponin and/or TLR4 agonist are formulated with a liposomal carrier.

Clause 52. The composition, kit, saponin, TLR4 agonist, adjuvant, method or use according to clause 51, wherein the saponin and/or TLR4 agonist are formulated with a liposomal carrier comprising a sterol, such as cholesterol.

Clause 53. The composition, kit, saponin, TLR4 agonist, adjuvant, method or use according to any one of clauses 1 to 53, wherein administration is repeated every week to every 6 months, such as every 2 weeks to every 6 weeks, such as every 4 weeks.

Clause 54. The composition, kit, saponin, TLR4 agonist, adjuvant, method or use according to any one of clauses 1 to 53, wherein administration is intramuscularly with a composition comprising VLPs containing 10 ug pp65 protein, and an adjuvant comprising 50 ug of QS21 and 50 ug of 3D-MPL in a liposomal formulation and administration is repeated every 4 weeks.

### EXAMPLES

The following examples describe some exemplary modes of making and practicing the immunogenic compositions that are described herein. It should be understood that these examples are for illustrative purposes only and are not meant to limit the scope of the compositions and methods described herein.

### Example 1

### Construction of DNA Expression Plasmids

This Example describes development of expression plasmids and constructs for expression of recombinant HCMV gene sequences (gB (SEQ ID NO: 10) and Gag/pp65 fusion gene (SEQ ID NO: 6 sequences). A standard expression plasmid generally consists of a promoter sequence of mammalian origin, an intron sequence, a PolyAdenylation signal sequence (PolyA), a pUC origin of replication sequence (pUC—pBR₃₂₂ is a colE1 origin/site of replication initiation and is used to replicate plasmid in bacteria such as E. coli (DH5a)), and an antibiotic resistance gene as a selectable marker for plasmid plaque selection. Within the plasmid following the intron are a variety of restriction enzyme sites that can be used to splice in a gene or partial gene sequence of interest.

The Propol II expression plasmid contains the pHCMV (early promoter for HCMV), a Beta-Globin Intron (BGL Intron), a rabbit Globin polyAdenylation signal sequence (PolyA), a pUC origin of replication sequence (pUC—pBR₃₂₂ is a colE1 origin/site of replication initiation and is used to replicate plasmid in bacteria such as E. coli (DH5a)), and an ampicillin resistance gene μ-lactamase (Amp R—selectable marker for plasmid confers resistance to ampicillin (100 μg/ml).

To develop a Gag MMLV expression construct (“MLV-Gag”), a complementary DNA (cDNA) sequence encoding a Gag polyprotein of MMLV (Gag without its C terminus Pol sequence) (Seq ID NO: 3) was cloned in a Propol II expression vector. To develop a gB expression construct (“gB”), the full-length sequence of gB was codon-optimized for human expression

(GenScript) (SEQ ID NO: 10) and was cloned in a Propol II expression vector including the extracellular portion, transmembrane domain (TM) and cytoplasmic portion (Cyto) of gB. To develop a Gag/pp65 expression construct (“Gag/pp65”), a sequence encoding the Gag polyprotein of MMLV (Gag without its C terminus Pol sequence) was fused with the full-length sequence of pp65 codon-optimized for human expression (GenScript) (SEQ ID NO: 6) and cloned in a Propol II expression vector.

DNA plasmids were amplified in competent E. coli (DH5a) and purified with endotoxin-free preparation kits according to standard protocols.

### Example 2

### Production of Virus-Like Particles

This Example describes methods for production of virus-like particles containing various recombinant HCMV antigens described in Example 1.

293SF-3F6 cell line derived from HEK 293 cells are a proprietary suspension cell culture grown in serum-free chemically defined media (CA 2,252,972 and US 6,210,922). The cells were transiently transfected using calcium phosphate methods with an MMLV-Gag /pp65 DNA expression plasmid and co-transfected with a gB DNA expression plasmid described in Example 1. Expression of HCMV antigens by the HEK 293 cells was confirmed by flow cytometry. After 48 to 72 hours of transfection, supernatants containing the VLPs were harvested and filtered through 0.45 um pore size membranes and further concentrated and purified by ultracentrifugation through a 20% sucrose cushion in a SW32 Beckman rotor (25,000 rpm, 2 hours, 4° C.). Pellets were resuspended in sterile endotoxin-free PBS (GIBCO) to obtain 500 times concentrated VLP stocks. Total protein was determined on an aliquot by a Bradford assay quantification kit (BioRad). Purified VLPs were stored at −80° C. until used. Each lot of purified VLPs was analyzed for the expression of gB, and MMLV-Gag/pp65 fusion protein by SDS-Page and Western Blot with specific antibodies to gB (CH 28 mouse monoclonal antibody to gB; Virusys Corporation; Pereira, Let al. 1984 Virology 139:73-86), and pp65 (CH12 mouse monoclonal antibody to UL83/pp65; Virusys Corporation; Pereira, L. et al. 1982 Infect Immun 36: 924-932). Antibodies were detected using enhanced chemilluminescence (ECL).

### Example 3

### Stimulation of T cells in PBMCs from Healthy HCMV-positive Subjects using gB/pp65Gag VLPs

The objective of this study was to evaluate the ability of gB/pp65Gag VLPs produced as described in Example 2 and purified by sucrose cushion ultracentrifugation to activate pre-existing HCMV-specific CD4+and CD8+ T cells in peripheral blood mononuclear cells (“PBMCs”) from healthy HCMV-positive subjects.

Human peripheral blood was obtained from CMV+healthy donors. PBMCs were isolated from whole blood using Ficoll gradient separation and single use aliquots were created. PBMCs were used either fresh after separation or after storage at −170° C. Briefly, PBMCs were cultured at 1 x 10⁶ cells/mL in 4 mL PP culture tubes. gB/pp65 eVLPs and controls were added to the cells. Cells were cultured for 3 hours with stimulating agents prior to addition of Monensin and cultured for an additional 10 hours.

Potency was evaluated in ex vivo PBMC cultures in terms of the frequency of IFN-gamma-secreting CD4+and CD8+ T cells. Cells were collected and stained for surface antigens using PerCP-conjugated anti-CD3, PE-conjugated anti-CD4, and APC-conjugated anti-CD8 monoclonal antibodies. Cells were then permeabilized and fixed for intracellular staining with BV510-conjugated anti-IFN-gamma. Stained wells were analyzed by flow cytometry analysis on a FACS Accuri (Beckton-Dickinson). Using FlowJo software (TreeStar), gating was first performed on CD3+cells to evaluate the proportion of IFN-gamma secreting cells among either the CD3+CD4+or the CD3+CD8+populations.

Data are shown in Table 1 below. The data are shown as mean percentage of cells, after subtraction of background responses to stimulation with empty VLPs (in the case of compositions comprising gB/pp65 VLPs) or unstimulated cells (in the case of recombinant proteins).

TABLE 1

| | Mean % IFN-gamma | Mean % IFN-gamma |
|---|---|---|
| | +ve T cells within | +ve T cells within |
| Composition | CD4+ population | CD8+ population |
| gB/pp65Gag VLP | 0.4811 | 0.8422 |
| (2 ug/ml gB content) | | |
| gB/pp65Gag VLP | 0.4511 | 1.537 |
| (1 ug/ml gB content) | | |
| Recomb gB (2 ug/ml) + | 0.0656 | 0.4789 |
| pp65* | | |
| Recomb gB (1 ug/ml) + | 0.0889 | 0.8433 |
| pp65* | | |

*pp65 content is not indicated as the experiment predates development of a validated pp65 ELISA

As shown above in Table 1, the bivalent gB/pp65Gag VLPs stimulate both CD4+and CD8+ IFN-gamma-secreting T cell responses ex vivo. A combination of recombinant gB and pp65 proteins was less effective than the bivalent VLPs at stimulating CD8+and particularly CD4+ T cell responses in the PBMCs from healthy subjects.

### Example 4

### Clinical Trial of gB/pp65Gag VLPs in Human GBM patients

The gB/pp65Gag VLPs will be tested in a Phase I/II study to define the safety, tolerability and impact of an immunogenic composition comprising gB/pp65Gag VLPs (10 ug gB/pp65Gag VLP based on pp65 protein content) formulated with liposomal adjuvant comprising QS21 (50 ug per dose) and 3D-MPL (50 ug per dose) on GBM patients.

Adult subjects (18-70 years of age) with their first recurrence of GBM with a tumour size that is 400mm² or less will participate. Patients will previously have undergone surgical tumour debulking followed by radiation and temozolomide and corticosteroids. Each subject will be administered the test treatment, intramuscularly, every 4 weeks.

Clinical disease progression is monitored by measurement of tumour size using MRI.

Response to the investigational drug is determined as follows:
Antibody titers against HCMV gB antigen by ELISA assay at baseline and 2 weeks after each treatment with results provided as serum IgG anti-gB antibody titers in baseline and post injection samples.
cellular immunity against HCMV gB and pp65 antigens using IFN-γ and IL-5 ELISPOT assessed at baseline and 2 weeks after each treatment. Results are provided as frequencies of IFN-γ and IL-5 spots per 3x10⁵ plated PBMCs post HCMV stimulation at baseline and after each treatment.
Progression free survival (PFS) from date of first dose to date of progression (or death.

Cellular immunity (CMI) data is expressed as spot forming cells (SFC) per 10⁶ PBMCs.

Tumour response is shown as SD (stable disease) or PD (progressed disease).

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

The application of which this description and claims forms part may be used as a basis for priority in respect of any subsequent application. The claims of such subsequent application may be directed to any feature or combination of features described herein. Embodiments are envisaged as being independently, fully combinable with one another where appropriate to the circumstances to form further embodiments of the invention. They may take the form of product, composition, process, or use claims and may include, by way of example and without limitation, the claims which follow.

All publications, including but not limited to patents and patent applications, cited in this specification are herein incorporated by reference as if each individual publication were specifically and individually indicated to be incorporated by reference herein as though fully set forth.

### Bibliography

Baryawno N et al., Detection of human cytomegalovirus in medulloblastomas reveals a potential therapeutic target J Clin Invest 2011 121, 4043-4055

Cloughesy et al., Neoadjuvant anti-PD-1 immunotherapy promotes a survival benefit with intratumoral and systemic immune responses in recurrent glioblastoma. Nature Medicine 2019 25: 477-486

Cobbs CS et al, Human cytomegalovirus infection and expression in human malignant glioma. Cancer Res. 2002 Jun 15;62(12):3347-50

Cobbs C et al, HCMV glycoprotein B is expressed in primary glioblastomas and enhances growth and invasiveness via PDGFR-alpha activation Oncotarget 2014; 5:1091-1100

Dziurzynski et al., Consensus on the role of human cytomegalovirus in glioblastoma Neuro-Onc. 2012 14:246-55

Gargon N and Di Pasquale A, From discovery to licensure, the Adjuvant System story. Human Vaccines & Immunotherapeutics 2017 13:1, 19-33, DOI: 10.1080/21645515.2016.1225635

Garrone et al., A Prime-Boost Strategy Using Virus-Like Particles Pseudotyped for HCV Proteins Triggers Broadly Neutralizing Antibodies in Macaques. Science Transl. Med. 2011 3: 1-8

Han et al 2014 Characterization of the Structure and Immunostimulatory Activity of a Vaccine Adjuvant, De-O-Acylated Lipooligosaccharide PLoS ONE 9(1):e85838 (and associated correction Han et al 2014 PLoS ONE 9(4):94517)

Harkins L E et al., Detection of human cytomegalovirus in normal and neoplastic breast epithelium Herpesviridae 2010 1(1):8

Libard S et al., Human Cytomegalovirus Tegument Protein pp65 Is Detected in All Intra- and Extra-Axial Brain Tumours Independent of the Tumour Type or Grade PLoS ONE 2014 9, e108861

Liu et al., Epstein-Barr virus- and cytomegalovirus-specific immune response in patients with brain cancer. J. Trans. Med., 2018 16:182

Mitchell DA et al., Sensitive detection of human cytomegalovirus in tumors and peripheral blood of patients diagnosed with glioblastoma. Neuro Oncol. 2008 Feb;10(1):10-8

Nair S K et al., Recognition and Killing of Autologous Primary Glioblastoma Tumor Cells by Human Cytomegalovirus pp65-Specific Cytotoxic T Cells. Clin Cancer Res 2014; 20: 2684-2694

Nauclér C S et al., The emerging role of human cytomegalovirus infection in human carcinogenesis: a review of current evidence and potential therapeutic implications. Oncotarget. 2019 Jul 2;10(42):4333-4347

Rahbar, A et al., Low levels of Human Cytomegalovirus Infection in Glioblastoma multiforme associates with patient survival - a case-control study. Herpesviridae 2012; 3:3

Rahbar A et al., Discordant humoral and cellular immune responses to Cytomegalovirus (CMV) in glioblastoma patients whose tumors are positive for CMV. Oncoimmunology 2015 Feb; 4(2): e982391

Schuessler A et al., Autologous T-cell Therapy for Cytomegalovirus as a Consolidative Treatment for Recurrent Glioblastoma. Cancer Res. 2014; 74: 3466-3476

Sharma S et al., Noninfectious virus-like particles produced by Moloney murine leukemia virus-based retrovirus packaging cells deficient in viral envelope become infectious in the presence of lipofection reagents. Proc Natl Acad Sci U S A. 1997;94:10803-8

Taher C et al., High prevalence of human cytomegalovirus proteins and nucleic acids in primary breast cancer and metastatic sentinel lymph nodes 2013 PLoS One 2013;8(2):e56795

Wolmer-Solberg N et al., Frequent detection of human cytomegalovirus in neuroblastoma: a novel therapeutic target? Int J Cancer 2013133, 2351-61

Woroniecka et al., T-Cell Exhaustion Signatures Vary with Tumor Type and Are Severe in Glioblastoma Clin Cancer Res. 2018 24 41 75-41 86

1. An immunogenic composition comprising:
(i) a virus like particle (VLP) comprising:
(a) a murine leukemia virus (MLV) gag protein;
(b) an HCMV pp65 protein; and
(c) an HCMV glycoprotein B (gB) protein;
and
(ii) an adjuvant comprising a saponin and a TLR4 agonist.

2. A kit for the preparation of an immunogenic composition, said kit comprising:
(i) a first container comprising a virus like particle comprising:
(a) a murine leukemia virus (MLV) gag protein;
(b) an HCMV pp65 protein; and
(c) an HCMV glycoprotein B (gB) protein;
and
(ii) a second container comprising an adjuvant comprising a saponin and a TLR4 agonist.

3. A method for eliciting an immune response in a subject, said method comprising the administration of a saponin, a TLR4 agonist and a virus like particle comprising:
(a) a murine leukemia virus (MLV) gag protein;
(b) an HCMV pp65 protein; and
(c) an HCMV glycoprotein B (gB) protein.

4. The method according to claim 3, wherein the saponin, the TLR4 agonist and the virus like particle are administered in the form of an immunogenic composition comprising:
(i) a virus like particle (VLP) comprising:
(a) a murine leukemia virus (MLV) gag protein;
(b) an HCMV pp65 protein; and
(c) an HCMV glycoprotein B (gB) protein;
and
(ii) an adjuvant comprising a saponin and a TLR4 agonist.

5. A method for the treatment of an HCMV associated cancer in a subject, said method comprising the administration of a saponin, a TLR4 agonist and a virus like particle comprising:
(a) a murine leukemia virus (MLV) gag protein;
(b) an HCMV pp65 protein; and
(c) an HCMV glycoprotein B (gB) protein.

6. The method according to claim 5, wherein the VLP comprises a MLV Gag polypeptide comprising an amino acid sequence at least 80% identical to SEQ ID NO:1.

7. The method according to any claim 5, wherein the VLP comprises a gB protein comprising an amino acid sequence which is at least 80% identical to SEQ ID NO: 8.

8. The method according to claim 5, wherein the VLP comprises a pp65 protein comprising an amino acid sequence which is at least 80% identical to SEQ ID NO: 11.

9. The method according to claim 5, wherein the VLP comprises a fusion protein comprising an amino acid sequence at least 80% identical to SEQ ID NO:4.

10. The method according to claim 5, wherein the saponin is QS21.

11. The method according to claim 5, wherein the TLR4 agonist is 3-de-O-acylated monophosphoryl lipid A (3D-MPL).

12. The method according to claim 5, wherein the subject is human.

13. The method according to claim 5, for the treatment of an HCMV associated cancer selected from breast, colon, ovarian and prostate cancer, rhabdomyosarcoma, hepatocellular cancer, salivary gland tumours, neuroblastoma and brain tumours.

14. The method according to claim 5, for the treatment of GBM.

15. The method according to claim 14, for use in the treatment of GBM in a subject experiencing their first occurrence.

16. The method according to claim 5, for use in the treatment of GBM in a subject having a tumour with a maximum cross-sectional area of 400 mm² or less.

17. The method according to claim 12, wherein the subject has a CD4/CD8 ratio of at least 2 at initiation of treatment.

18. The method according to claim 17, wherein the subject has a CD4/CD8 ratio of at least 3 at initiation of treatment.

19. The method according to claim 12, wherein the subject has a CD4/CD8 ratio of less than 3 at initiation of treatment.

20. The method according to claim 5, wherein a human dose comprises a gB protein content of 1/200^{th} to 1/10^{th} of content of pp65, such as 1/120^{th} to 1/40^{th} of content of pp65, in particular 1/100^{th} to 1/60^{th} of content of pp65 on a weight basis.

21. The method according to claim 5, wherein administration is intramuscularly with a composition comprising VLPs containing 10 ug pp65 protein, and an adjuvant comprising 50 ug of QS21 and 50 ug of 3D-MPL in a liposomal formulation and administration is repeated every 4 weeks.
